(19) United States
(12) Patent Application Publication
(10) Pub. No.: US 2026/0295031 A1
(43) Pub. Date: Oct. 1, 2026

(54) IMMUNOGENIC COMPOSITION AND USES THEREOF

(71) Applicant: University of Florida Research Foundation, Inc., Gainesville, FL (US)

(72) Inventors: Janet K. Yamamoto, Gainesville, FL (US); Subhashinie Kariyawasam, Gainesville, FL (US); Bikash Sahay, Gainesville, FL (US)

(21) Appl. No.: 19/100,972

(22) PCT Filed: Aug. 25, 2023
(86) PCT No.: PCT/US23/72907

§ 371 Date: Feb. 4, 2025

Related U.S. Application Data
(60) Provisional application No. 63/373,474, filed on Aug. 25, 2022.
(60) Provisional application No. 63/508,697, filed on Jun. 16, 2023.

(51) Int. Cl. A61K 39/215; A61K 39/00; A61P 37/04

(52) U.S. Cl. CPC A61K 39/215; A61P 37/04; A61K 2039/53

(57) ABSTRACT

The disclosure provides a composition comprising a feline coronavirus (FCoV) receptor binding domain peptide and a human severe acute respiratory syndrome coronavirus serotype 2 (SCoV2) receptor domain peptide and uses thereof.

### FIELD

The present disclosure is in the field of vaccines and the treatment or prevention of coronavirus infections.

### INCORPORATION BY REFERENCE OF MATERIALS SUBMITTED ELECTRONICALLY

This application contains, as a separate part of the disclosure, a Sequence Listing in computer readable form (Filename: 57693_SeqListing.xml; Size: 92,077 bytes; Created Aug. 24, 2023), which is incorporated by reference in its entirety.

### BACKGROUND

Coronaviruses (CoVs) are a highly diverse family of enveloped positive-sense, single-stranded RNA viruses with a lipid envelope. CoVs infect humans, other mammals including livestock and companion animals and avian species like domestic and free-living birds. Feline (FCoV) and canine (CCoV) coronaviruses are widespread among dog and cat populations. FCoV belongs to the alphacoronavirus genus and two phylogenetic lineages are currently known, serotype 1 and 2 (FCoV1, FCoV2). Recombination is a common behavior for CoVs that may generate new variants able to infect species other than their natural reservoir.¹ For example, FCoV2 originated by a double recombination event between the FCoV1 and CCoV.²

Both FCoV serotypes infect predominantly epithelial cells of the gastrointestinal tract and cause a mild gastrointestinal disease (e.g., diarrhea, vomiting, and transient weight loss) in domestic cats and especially in kittens. Often, upon chronic infection, these viruses can also mutate into pathogenic and fatal variants. These variants, called feline infectious peritonitis viruses (FIPVs), infect monocytes and macrophages, leading to systemic infection³⁻⁵. No successful FIPV treatments have been available for decades; cats with FIPV either die or have to be euthanized.⁶ The median survival time of untreated cats is only eight to nine days.⁶ Unfortunately, FIPV vaccine development has been hampered by antibody dependent enhancement (ADE), whereby vaccine candidates that induce neutralizing antibodies make the infection worse.⁷⁻⁹

For humans, three highly pathogenic and deadly human coronaviruses have emerged within the last two decades, namely SARS-CoV1 (SCoV1), MERS-CoV and the virus causing the current COVID-19 pandemic, SARS-CoV-2 (SCoV2).¹⁰ Unlike the FCoVs, SCoV1 and SCoV2 belong to the betacoronavirus genus. SCoV2 is thought to have originated from recombination events between CoVs of other species, possibly through one or more intermediate hosts.^{11, 12} The COVID-19 pandemic has been partially controlled using SARS-CoV-2 vaccines. However, while the vaccines prevent severe and fatal disease, their efficacy wanes over time, necessitating booster doses.¹³ Vaccination has also been unable to prevent “breakthrough” infections in fully vaccinated individuals, allowing subsequent transmission of the disease to others.¹³ Moreover, immunity following natural infection with SARS-CoV-2, combined with vaccine-induced immunity, has so far not prevented the emergence and rapid spread of “variants of concern,” such as the highly transmissible delta (B.1.617.2) variant and the omicron (B.1.1.529) variant.¹³

There is now evidence of SARS-CoV-2 spillover from humans to animals reported in dogs, cats, tigers, lions, gorillas and minks.¹⁴⁻¹⁶ SARS-CoV-2 infection in domestic dogs and cats has been reported in 22 countries in America, Europe and Asia.^{17,18} Cats are susceptible to SARS-CoV-2 infection and can show mild-to-moderate respiratory symptoms, while dogs developed no or mild respiratory symptoms.^{14,19,20} There remains a need in the art for a vaccine that is protective across multiple species.

### SUMMARY

In one aspect, the disclosure provides a composition comprising nucleic acid encoding a feline coronavirus (FCoV) receptor binding domain (RBD) peptide and a severe acute respiratory syndrome coronavirus 2 (SCoV2) RBD peptide.

In another aspect, the disclosure provides methods of stimulating an immune response in a subject in need there comprising administering to the subject a composition comprising nucleic acid encoding a feline coronavirus (FCoV) RBD peptide and a severe acute respiratory syndrome coronavirus 2 (SCoV2) RBD peptide.

### BRIEF DESCRIPTION OF THE DRAWINGS

FIG. 1 top panel provides the UF-RBD SCoV2 sequence (SEQ ID NO: 1). The middle panel provides the MB-RBD SCoV2 sequence (SEQ ID NO: 3). The bottom panel provides a sequence alignment of both sequences showing that the MB-RBD sequence is longer by 12 aa on the carboxyl-end.

FIG. 2A is a bar graph of FCoV2 antibody titers generated by ELISA using FCoV2 whole-virus as the antigen, illustrating FCoV2 seropositivity of four naturally infected laboratory queens (UGAQ1, UGAQ2, UGAQ3, UGAQ4) and weak FCoV2 seropositivity of sera obtained from three previously SPF toms (5HQT1, HOJT2, HOGT3) after they mated with the positive queens. The bars filled with the slashed lines depict the mating pair, UGAQ3 and 5HQT1 and the resulting juvenile cat, Y2B. The bars filled with small dots represent the mating pair UGAQ4 and HOJT2, and their litter D4A, D4D, and D4F. The unfilled bars show two queens (UGAQ1, UGAQ2) and a tom (HOGT3) that did not successfully mate. The serum from SPF cat, HOE, was used as a negative control. FIG. 2B is a bar graph of antibody titers generated by ELISA using SCoV2 RBD illustrating cross-reactivity of the FCoV2 anti-sera obtained from the queens and juvenile cats with SCoV2 RBD. Serum from SPF cat, was used as a negative control. The month(s) post UF arrival when the blood collection were performed on the queens.

FIG. 3A is a bar graph showing antibody titers, generated under stringent ELISA conditions, against FCoV2 (SEQ ID NO: 5), SCoV2-UF-RBD (SEQ ID NO: 1), SCoV2-MB-RBD (SEQ ID NO: 3) and bovine serum albumin (BSA) using anti-sera from FCoV positive Queens. Serum from HOE SPF was used as a negative control. The ages of the queens is indicated.

FIG. 3B is a bar graph showing antibody titers, generated under stringent ELISA conditions, against FCoV2, UF-RBD, and MB-RBD using sera obtained from toms before and after mating with FCoV positive queens.

FIG. 4 is a bar graph showing lack of bovine serum albumin (BSA) antibodies in sera from the three toms pre-mating and at two time points post mating with the FCoV positive queens.

FIG. 5A is a bar graph whose first three sets of bars show antibody titers obtained under stringent ELISA conditions, against FCoV2 whole virus antigen, SCoV2 RBD, and bovine serum albumin (BSA) using sera obtained from the juvenile cats. The dark bars indicate titers from sera obtained at 12 weeks of age for Y2B and at 16 weeks of age for D4A, D4D, and D4F. The light bars indicate titers from sera obtained at 16 weeks of age for Y2B and at 20 weeks of age for the three D4 cats. Serum from HOE SPF was used as a negative control while serum from FCoV positive queen (UGAQ4) was used as a positive control.

FIGS. 5B1 and B2 are images showing immunoblot strips results for each antigen antibody titer group described in (FIG. 5A) with the exception of the BSA antigen group which was not performed. The serum from SPF cat HOE was used as negative controls for all three antigens.

FIGS. 6A-6D provide immunoblot analyses results of the sera from FCoV+ queens and SCoV2 RBD-positive toms using the SCoV2 UF-RBD (FIG. 6A), the proposed FCoV2 RBD (FIG. 6B), the cross-reactive FCoV2-wv (FIG. 6C), and the proposed (FIG. 6D) FCoV1 RBD immunoblot strips.

FIGS. 7A and 7B are graphs showing the anti-CoV RBD IFNγ and IL-2 ELISpot responses of the peripheral blood mononuclear cells (PBMC) from a COVID-19 vaccinated subjects.

### DETAILED DESCRIPTION

The present disclosure is based, in part, on the discovery of compositions comprising a nucleic acid encoding a feline coronavirus (FCoV) receptor binding domain (RBD) peptide and a human coronavirus serotype 2 (SCoV2) RBD peptide for the prevention and/or treatment of FCoV1, FCoV2, CCoV and SCoV2 coronavirus infections. As shown in the Examples, the disclosed composition induced strong immune responses in subjects immunized with the compositions.

In some or any aspects, the composition comprising a nucleic acid or peptide disclosed herein elicits an immune response in the subject when administered to the subject. In some embodiments, the compositions may prevent, ameliorate, palliate, or eliminate disease from the host.

### Coronavirus

The term “coronavirus” refers to a virus in the family Coronaviridae, which is in turn classified within the order Nidovirales. The coronaviruses are large, enveloped, positive-stranded RNA viruses. The coronaviruses have the largest genomes of the RNA viruses known in the art and replicate by a unique mechanism that results in a high frequency of recombination. The coronaviruses include antigenic groups I, II, and III. Nonlimiting examples of coronaviruses include SARS coronavirus (e.g., SARS-CoV and SARS-CoV-2), MERS coronavirus, transmissible gastroenteritis virus (TGEV), human respiratory coronavirus, porcine respiratory coronavirus, canine coronavirus, feline enteric coronavirus, feline infectious peritonitis virus, rabbit coronavirus, murine hepatitis virus, sialodacryoadenitis virus, porcine hemagglutinating encephalomyelitis virus, bovine coronavirus, avian infectious bronchitis virus, and turkey coronavirus, as well as chimeras thereof. Additional information related to coronavirus including classification, virion structure, genome structure, genetics and pathology is described, for example, in KV Holmes, Encyclopedia of Virology, 1999:291-298, the content of which is incorporated herein by reference.

In some or any aspects, a coronavirus described herein is in the genus of Alpha-coronavirus, and the coronavirus antigens can be of or derived from any species or strains in the genus of Alpha-coronavirus. In some or any aspects, a coronavirus described herein is in the genus of Beta-coronavirus and the coronavirus antigens can be of or derived from any species or strains in the genus of Beta-coronavirus. Member viruses in the genus of Alphacoronavirus and Beta-coronavirus are enveloped, positive-strand RNA viruses that can infect mammals.

A coronavirus described herein is optionally in the subgenus Tegacovirus of Alpha-coronavirus genus, including but not limited to feline coronavirus serotype 1 (FCoV1), feline coronavirus serotype 2 (FCoV2) and canine coronavirus (CCoV).

Within the genus Beta-coronavirus, five subgenera or lineages have been recognized, including Embecovirus (lineage A), Sarbecovirus (lineage B), Merbecovirus (lineage C), Nobecovirus (lineage D), and Hibecovirus. Accordingly, in some or any aspects, the coronavirus described herein can be any strain or species in any of the subgenera or lineages of Beta-coronavirus. For example, a coronavirus peptide can be of or derived from any species or strains in the subgenus of Sarbecovirus, including but not limited to human SARS-CoV and SARS-CoV2.

In some or any aspects, the compositions described herein comprises a nucleic acid encoding a feline coronavirus (FCoV) receptor binding domain (RBD) peptide and a human Severe acute respiratory syndrome (SARS) coronavirus 2 (SCoV2) protein RBD peptide.

In some or any aspects, the FCoV RBD peptide is an FCoV serotype 1 (FCoV1) RBD peptide. In some or any aspects, the nucleic acid encodes a FCoV1 RBD peptide comprising an amino acid sequence that is at least 70% identical to the amino acid sequence set forth in SEQ ID NO: 5. In some or any aspects, the nucleic acid encodes a FCoV1 RBD peptide comprising an amino acid sequence that is at least 70% (e.g., at least 80%, 85%, 90%, 91%, 92%, 93%, 94%, 95%, 96%, 97%, 98%, 99% or more) identical to the amino acid sequence set forth in SEQ ID NO: 5. In some or any aspects, the nucleic acid encodes a FCoV1 RBD peptide comprising the amino acid sequence set forth in SEQ ID NO: 5.

In some or any aspects, the FCoV RBD peptide is an FCoV serotype 2 (FCoV2) RBD peptide. In some or any aspects, the nucleic acid encodes a FCoV2 RBD peptide comprising an amino acid sequence that is at least 70% identical to the amino acid sequence set forth in SEQ ID NO: 7. In some or any aspects, the nucleic acid encodes a FCoV2 RBD peptide comprising an amino acid sequence that is at least 70% (e.g., at least 80%, 85%, 90%, 91%, 92%, 93%, 94%, 95%, 96%, 97%, 98%, 99% or more) identical to the amino acid sequence set forth in SEQ ID NO: 7. In some or any aspects, the nucleic acid encodes a FCoV2 RBD peptide comprising the amino acid sequence set forth in SEQ ID NO: 7.

In some or any aspects, the nucleic acid encodes a SCoV2 RBD peptide comprising an amino acid sequence that is at least 70% identical to the amino acid sequence set forth in SEQ ID NO: 1 (UF-RBD). In some or any aspects, the composition comprises a SCoV2 RBD peptide comprising an amino acid sequence that is at least 70% (e.g., at least 80%, 85%, 90%, 91%, 92%, 93%, 94%, 95%, 96%, 97%, 98%, 99% or more) identical to the amino acid sequence set forth in SEQ ID NO: 1. In some or any aspects, the nucleic acid encodes a SCoV2 RBD peptide comprising the amino acid sequence set forth in SEQ ID NO: 1.

In some or any aspects, the nucleic acid encodes a SCoV2 RBD peptide comprising an amino acid sequence that is at least 70% identical to the amino acid sequence set forth in SEQ ID NO: 3 (MB-RBD). In some or any aspects, the nucleic acid encodes a SCoV2 RBD peptide comprising an amino acid sequence that is at least 70% (e.g., at least 80%, 85%, 90%, 91%, 92%, 93%, 94%, 95%, 96%, 97%, 98%, 99% or more) identical to the amino acid sequence set forth in SEQ ID NO: 3. In some or any aspects, the nucleic acid encodes a SCoV2 RBD peptide comprising the amino acid sequence set forth in SEQ ID NO: 3.

In some or any aspects, the nucleic acid encodes a SCoV2 RBD peptide comprising an amino acid sequence that is at least 70% identical to the amino acid sequence set forth in SEQ ID NO: 9. In some or any aspects, the nucleic acid encodes a SCoV2 RBD peptide comprising an amino acid sequence that is at least 70% (e.g., at least 80%, 85%, 90%, 91%, 92%, 93%, 94%, 95%, 96%, 97%, 98%, 99% or more) identical to the amino acid sequence set forth in SEQ ID NO: 9. In some or any aspects, the nucleic acid encodes a SCoV2 RBD peptide comprising the amino acid sequence set forth in SEQ ID NO: 9.

In some or any aspects, the nucleic acid encodes a SCoV2 RBD peptide comprising an amino acid sequence that is at least 70% identical to the amino acid sequence set forth in SEQ ID NO: 10. In some or any aspects, the nucleic acid encodes a SCoV2 RBD peptide comprising an amino acid sequence that is at least 70% (e.g., at least 80%, 85%, 90%, 91%, 92%, 93%, 94%, 95%, 96%, 97%, 98%, 99% or more) identical to the amino acid sequence set forth in SEQ ID NO: 10. In some or any aspects, the nucleic acid encodes a SCoV2 RBD peptide comprising the amino acid sequence set forth in SEQ ID NO: 10.

In some or any aspects, the nucleic acid encodes a FCoV1 RBD peptide comprising an amino acid sequence that is at least 70% identical to the amino acid sequence set forth in SEQ ID NO: 5 and a SCoV2 RBD peptide comprising an amino acid sequence that is at least 70% identical to the amino acid sequence set forth in SEQ ID NO. 1. In some or any aspects, the nucleic acid encodes a FCoV1 RBD domain peptide comprising an amino acid sequence that is at least 70% (e.g., at least 80%, 85%, 90%, 91%, 92%, 93%, 94%, 95%, 96%, 97%, 98%, 99% or more) identical to the amino acid sequence set forth in SEQ ID NO: 5 and a SCoV2 RBD peptide comprising an amino acid sequence that is at least 70% (e.g., at least 80%, 85%, 90%, 91%, 92%, 93%, 94%, 95%, 96%, 97%, 98%, 99% or more) identical to the amino acid sequence set forth in SEQ ID NO:1. In some or any aspects, the nucleic acid encodes a FCoV1 RBD peptide comprising the amino acid sequence set forth in SEQ ID NO: 5 and a SCoV2 RBD peptide comprising the amino acid sequence set forth in SEQ ID NO: 1.

In some embodiments, the nucleic acid encodes a FCoV1 RBD peptide comprising an amino acid sequence that is at least 70% identical to the amino acid sequence set forth in SEQ ID NO: 5 and a SCoV2 RBD peptide comprising an amino acid sequence that is at least 70% identical to the amino acid sequence set forth in SEQ ID NO: 3. In some or any aspects, the nucleic acid encodes a FCoV1 RBD domain peptide comprising an amino acid sequence that is at least 70% (e.g., at least 80%, 85%, 90%, 91%, 92%, 93%, 94%, 95%, 96%, 97%, 98%, 99% or more) identical to the amino acid sequence set forth in SEQ ID NO: 5 and a SCoV2 RBD peptide comprising an amino acid sequence that is at least 70% (e.g., at least 80%, 85%, 90%, 91%, 92%, 93%, 94%, 95%, 96%, 97%, 98%, 99% or more) identical to the amino acid sequence set forth in SEQ ID NO: 3. In some or any aspects, the nucleic acid encodes a FCoV1 RBD peptide comprising the amino acid sequence set forth in SEQ ID NO: 5 and a SCoV2 RBD peptide comprising the amino acid sequence set forth in SEQ ID NO: 3.

In some embodiments, the nucleic acid encodes a FCoV1 RBD peptide comprising an amino acid sequence that is at least 70% identical to the amino acid sequence set forth in SEQ ID NO: 5 and a SCoV2 RBD peptide comprising an amino acid sequence that is at least 70% identical to the amino acid sequence set forth in SEQ ID NO: 9. In some or any aspects, the nucleic acid encodes a FCoV1 RBD domain peptide comprising an amino acid sequence that is at least 70% (e.g., at least 80%, 85%, 90%, 91%, 92%, 93%, 94%, 95%, 96%, 97%, 98%, 99% or more) identical to the amino acid sequence set forth in SEQ ID NO: 5 and a SCoV2 RBD peptide comprising an amino acid sequence that is at least 70% (e.g., at least 80%, 85%, 90%, 91%, 92%, 93%, 94%, 95%, 96%, 97%, 98%, 99% or more) identical to the amino acid sequence set forth in SEQ ID NO. 9. In some or any aspects, the nucleic acid encodes a FCoV1 RBD peptide comprising the amino acid sequence set forth in SEQ ID NO: 5 and a SCoV2 RBD peptide comprising the amino acid sequence set forth in SEQ ID NO: 9.

In some embodiments, the nucleic acid encodes a FCoV1 RBD peptide comprising an amino acid sequence that is at least 70% identical to the amino acid sequence set forth in SEQ ID NO: 5 and a SCoV2 RBD peptide comprising an amino acid sequence that is at least 70% identical to the amino acid sequence set forth in SEQ ID NO: 10. In some or any aspects, the nucleic acid encodes a FCoV1 RBD domain peptide comprising an amino acid sequence that is at least 70% (e.g., at least 80%, 85%, 90%, 91%, 92%, 93%, 94%, 95%, 96%, 97%, 98%, 99% or more) identical to the amino acid sequence set forth in SEQ ID NO: 5 and a SCoV2 RBD peptide comprising an amino acid sequence that is at least 70% (e.g., at least 80%, 85%, 90%, 91%, 92%, 93%, 94%, 95%, 96%, 97%, 98%, 99% or more) identical to the amino acid sequence set forth in SEQ ID NO: 10. In some or any aspects, the nucleic acid encodes a FCoV1 RBD peptide comprising the amino acid sequence set forth in SEQ ID NO: 5 and a SCoV2 RBD peptide comprising the amino acid sequence set forth in SEQ ID NO: 10.

In some or any aspects, the nucleic acid encodes a FCoV2 RBD peptide comprising an amino acid sequence that is at least 70% identical to the amino acid sequence set forth in SEQ ID NO: 7 and a SCoV2 RBD peptide comprising an amino acid sequence that is at least 70% identical to the amino acid sequence set forth in SEQ ID NO.: 1. In some or any aspects, the nucleic acid encodes a FCoV2 RBD peptide comprising an amino acid sequence that is at least 70% (e.g., at least 80%, 85%, 90%, 91%, 92%, 93%, 94%, 95%, 96%, 97%, 98%, 99% or more) identical to the amino acid sequence set forth in SEQ ID NO: 7 and a SCoV2 RBD peptide comprising an amino acid sequence that is at least 70% (e.g., at least 80%, 85%, 90%, 91%, 92%, 93%, 94%, 95%, 96%, 97%, 98%, 99% or more) identical to the amino acid sequence set forth in SEQ ID NO: 1. In some or any aspects, the nucleic acid encodes a FCoV2 RBD peptide comprising the amino acid sequence set forth in SEQ ID NO: 7 and a SCoV2 RBD peptide comprising the amino acid sequence set forth in SEQ ID NO: 1.

In some or any aspects, the nucleic acid encodes a FCoV2 RBD peptide comprising an amino acid sequence that is at least 70% identical to the amino acid sequence set forth in SEQ ID NO: 7 and a SCoV2 RBD peptide comprising an amino acid sequence that is at least 70% identical to the amino acid sequence set forth in SEQ ID NO: 3. In some or any aspects, the nucleic acid encodes a FCoV2 RBD peptide comprising an amino acid sequence that is at least 70% (e.g., at least 80%, 85%, 90%, 91%, 92%, 93%, 94%, 95%, 96%, 97%, 98%, 99% or more) identical to the amino acid sequence set forth in SEQ ID NO: 7 and a SCoV2 RBD peptide comprising an amino acid sequence that is at least 70% (e.g., at least 80%, 85%, 90%, 91%, 92%, 93%, 94%, 95%, 96%, 97%, 98%, 99% or more) identical to the amino acid sequence set forth in SEQ ID NO: 3. In some or any aspects, the nucleic acid encodes a FCoV2 RBD peptide comprising the amino acid sequence set forth in SEQ ID NO: 7 and a SCoV2 RBD peptide comprising the amino acid sequence set forth in SEQ ID NO: 3.

In some or any aspects, the nucleic acid encodes a FCoV2 RBD peptide comprising an amino acid sequence that is at least 70% identical to the amino acid sequence set forth in SEQ ID NO: 7 and a SCoV2 RBD peptide comprising an amino acid sequence that is at least 70% identical to the amino acid sequence set forth in SEQ ID NO: 9. In some or any aspects, the nucleic acid encodes a FCoV2 RBD peptide comprising an amino acid sequence that is at least 70% (e.g., at least 80%, 85%, 90%, 91%, 92%, 93%, 94%, 95%, 96%, 97%, 98%, 99% or more) identical to the amino acid sequence set forth in SEQ ID NO: 7 and a SCoV2 RBD peptide comprising an amino acid sequence that is at least 70% (e.g., at least 80%, 85%, 90%, 91%, 92%, 93%, 94%, 95%, 96%, 97%, 98%, 99% or more) identical to the amino acid sequence set forth in SEQ ID NO: 9. In some or any aspects, the nucleic acid encodes a FCoV2 RBD peptide comprising the amino acid sequence set forth in SEQ ID NO: 7 and a SCoV2 RBD peptide comprising the amino acid sequence set forth in SEQ ID NO: 9.

In some or any aspects, the nucleic acid encodes a FCoV2 RBD peptide comprising an amino acid sequence that is at least 70% identical to the amino acid sequence set forth in SEQ ID NO: 7 and a SCoV2 RBD peptide comprising an amino acid sequence that is at least 70% identical to the amino acid sequence set forth in SEQ ID NO: 10. In some or any aspects, the nucleic acid encodes a FCoV2 RBD peptide comprising an amino acid sequence that is at least 70% (e.g., at least 80%, 85%, 90%, 91%, 92%, 93%, 94%, 95%, 96%, 97%, 98%, 99% or more) identical to the amino acid sequence set forth in SEQ ID NO: 7 and a SCoV2 RBD peptide comprising an amino acid sequence that is at least 70% (e.g., at least 80%, 85%, 90%, 91%, 92%, 93%, 94%, 95%, 96%, 97%, 98%, 99% or more) identical to the amino acid sequence set forth in SEQ ID NO: 10. In some or any aspects, the nucleic acid encodes a FCoV2 RBD peptide comprising the amino acid sequence set forth in SEQ ID NO: 7 and a SCoV2 RBD peptide comprising the amino acid sequence set forth in SEQ ID NO: 10.

In some or any aspects, the comprises comprising a FCoV1 RBD peptide, a FCoV2 RBD peptide, and a SCoV2 RBD peptide. In some or any aspects, the nucleic acid encodes a FCoV1 RBD peptide comprising an amino acid sequence that is at least 70% identical to the amino acid sequence set forth in SEQ ID NO: 5; a FCoV2 RBD peptide comprising an amino acid sequence that is at least 70% identical to the amino acid sequence set forth in SEQ ID NO: 7, and a SCoV2 RBD peptide comprising an amino acid sequence that is at least 70% identical to the amino acid sequence set forth in SEQ ID NO: 1, SEQ ID NO: 3, SEQ ID NO: 9 or SEQ ID NO: 10. In some or any aspects, the nucleic acid encodes a FCoV1 RBD peptide comprising an amino acid sequence that is at least 70% (e.g., at least 80%, 85%, 90%, 91%, 92%, 93%, 94%, 95%, 96%, 97%, 98%, 99% or more) identical to the amino acid sequence set forth in SEQ ID NO: 5, a FCoV2 RBD peptide comprising an amino acid sequence that is at least 70% (e.g., at least 80%, 85%, 90%, 91%, 92%, 93%, 94%, 95%, 96%, 97%, 98%, 99% or more) identical to the amino acid sequence set forth in SEQ ID NO: 7, and a SCoV2 RBD peptide comprising an amino acid sequence that is at least 70% (e.g., at least 80%, 85%, 90%, 91%, 92%, 93%, 94%, 95%, 96%, 97%, 98%, 99% or more) identical to the amino acid sequence set forth in SEQ ID NO: 1, SEQ ID NO: 3, SEQ ID NO: 9, or SEQ ID NO: 10.

In some or any aspects, the nucleic acid encodes a FCoV1 RBD peptide comprising the amino acid sequence set forth in SEQ ID NO: 5, a FCoV2 RBD peptide comprising the amino acid sequence set forth in SEQ ID NO: 7, and a SCoV2 peptide comprising the amino acid sequence set forth in SEQ ID NO: 1. In some or any aspects, the nucleic acid encodes a FCoV1 RBD peptide comprising the amino acid sequence set forth in SEQ ID NO: 5, a FCoV2 RBD peptide comprising the amino acid sequence set forth in SEQ ID NO: 7, and a SCoV2 peptide comprising the amino acid sequence set forth in SEQ ID NO: 3. In some or any aspects, the nucleic acid encodes a FCoV1 RBD peptide comprising the amino acid sequence set forth in SEQ ID NO: 5, a FCoV2 RBD peptide comprising the amino acid sequence set forth in SEQ ID NO: 7, and a SCoV2 peptide comprising the amino acid sequence set forth in SEQ ID NO: 9. In some or any aspects, the nucleic acid encodes a FCoV1 RBD peptide comprising the amino acid sequence set forth in SEQ ID NO: 5, a FCoV2 RBD peptide comprising the amino acid sequence set forth in SEQ ID NO: 7, and a SCoV2 peptide comprising the amino acid sequence set forth in SEQ ID NO: 10.

In some or any aspects, the composition optionally further comprises a nucleic acid encoding a canine coronavirus serotype 1 (CCoV1) RBD peptide or a CCoV serotype 2 (CCoV2) RBD peptide.

In some or any aspects, the CCoV1 RBD peptide comprises an amino acid sequence that is at least 70% (e.g., at least 80%, 85%, 90%, 91%, 92%, 93%, 94%, 95%, 96%, 97%, 98%, 99% or more) identical to the amino acid sequence set forth in SEQ ID NO: 11. In some or any aspects, the CCoV1 RBD peptide comprises the amino acid sequence set forth in SEQ ID NO: 11.

In some or any aspects, the CCoV2 RBD peptide comprises an amino acid sequence that is at least 70% (e.g., at least 80%, 85%, 90%, 91%, 92%, 93%, 94%, 95%, 96%, 97%, 98%, 99% or more) identical to the amino acid sequence set forth in SEQ ID NO: 12. In some or any aspects, the CCoV2 RBD comprises the amino acid sequence set forth in SEQ ID NO: 12.

In some or any aspects, the composition comprises a nucleic acid that encodes an amino acid sequence that is at least 70% (e.g., at least 80%, 85%, 90%, 91%, 92%, 93%, 94%, 95%, 96%, 97%, 98%, 99% or more) identical to the amino acid sequence set forth in SEQ ID NO: 93. In some or any aspects, the composition comprises a nucleic acid that encodes an amino acid sequence set forth in SEQ ID NO: 93.

In some or any aspects, the composition comprises a nucleic acid that encodes an amino acid sequence that is at least 70% (e.g., at least 80%, 85%, 90%, 91%, 92%, 93%, 94%, 95%, 96%, 97%, 98%, 99% or more) identical to the amino acid sequence set forth in SEQ ID NO: 94. In some or any aspects, the composition comprises an amino acid sequence set forth in SEQ ID NO: 94.

In some or any aspects, the composition comprises a nucleic acid that encodes an amino acid sequence that is at least 70% (e.g., at least 80%, 85%, 90%, 91%, 92%, 93%, 94%, 95%, 96%, 97%, 98%, 99% or more) identical to the amino acid sequence set forth in SEQ ID NO: 95. In some or any aspects, the composition comprises a nucleic acid that encodes an amino acid sequence set forth in SEQ ID NO: 95.

A sequence which is not 100% identical to the particular sequences described herein (e.g., SEQ ID NO: 1, SEQ ID NO: 3, SEQ ID NO: 5, SEQ ID NO: 7, SEQ ID NO: 9, SEQ ID NO: 10, SEQ ID NO: 11, SEQ ID NO: 12, SEQ ID NO: 93, SEQ ID NO: 94, SEQ ID NO: 95, etc.) may comprise one or more conservative substitutions. Conservative substitutions generally correspond to substitution of a reference amino acid with a functionally equivalent reside with similar physiochemical properties. A functionally equivalent residue of an amino acid typically can refer to other amino acid residues having physiochemical and stereochemical characteristics substantially similar to the original amino acid. The physiochemical properties include water solubility (hydrophobicity or hydrophilicity), dielectric and electrochemical properties, physiological pH, partial charge of side chains (positive, negative or neutral) and other properties identifiable to one of skill in the art. The stereochemical characteristics include spatial and conformational arrangement of the amino acids and their chirality. For example, glutamic acid is considered to be a functionally equivalent residue to aspartic acid in the sense of the current disclosure. Tyrosine and tryptophan are considered as functionally equivalent residues to phenylalanine. Arginine and lysine are considered as functionally equivalent residues to histidine.

### Immunogen

In some or any aspects, the composition described herein comprises (or is administered with) an immunogenic epitope The term “immunogenic epitope as used herein refers to smallest component of an antigen that induces a subject to mount an immune response, and when epitopes are combined they serve as the vaccine immunogen. An antigen is composed of multiple epitopes to induce strong immune response. The terms “immunogenic epitope” and “immunogen” are used synonymously herein.

In some or any aspects, the “epitope” refers to a portion of antigen that an immunoglobulin or antibody binds to, e.g., at least 3, 4, 5, 6, 7, 8, 9, 10, 11, 12, 13, 14, 15, 20, 25, 30, 35, 40, 45, 50, 55, 60, 65, 70, 75 or more consecutive or non-consecutive amino acids in a unique steric conformation, which may be “linear” or “conformational”. See, for example, Epitope Mapping Protocols in Methods in Molecular Biology, Vol. 66, G. E. Morris, Ed. (1996). In a linear epitope, all the interaction sites between a protein and an interaction molecule (e.g., an antibody) are present linearly along the primary amino acid sequence of the protein. In a conformational epitope, the interaction sites span over amino acid residues that are not necessarily adjacent in the primary sequence but from a three dimensional structure recognized by an antibody.

An “immune response” is a response of a cell of the immune system, such as a B cell, T cell, or monocyte, to a stimulus, such as an antigen (e.g., formulated as an antigenic composition or a vaccine). An immune response can be a B cell response, which results in the production of specific antibodies, such as antigen specific neutralizing antibodies. An immune response can also be a T cell response, such as a CD4+ response or a CD8+ response. B cell and T cell responses are aspects of a “cellular” immune response. An immune response can also be a “humoral” immune response, which is mediated by antibodies. In some cases, the response is specific for a particular antigen (that is, an “antigen-specific response”). A “protective immune response” is an immune response that inhibits a detrimental function or activity of an antigen, or decreases symptoms (including death) that result from the antigen. A protective immune response can be measured, for example, by immune assays using a serum sample from an immunized subject for testing the ability of serum antibodies for inhibition of tumor cell expansion, such as: ELISA-neutralization assay, antibody dependent cell-mediated cytotoxicity assay (ADCC), complement-dependent cytotoxicity (CDC), antibody dependent cell-mediated phagocytosis (ADCP), enzyme-linked immunospot (ELISpot). In addition, vaccine efficacy can be tested by measuring the T cell response CD4+ and CD8+ after immunization, using flow cytometry (FACS) analysis or ELISpot assay. The protective immune response can be tested by measuring resistance to antigen challenge in vivo in an animal model. In humans, a protective immune response can be demonstrated in a population study, comparing measurements of symptoms, morbidity, mortality, etc. in treated subjects compared to untreated controls. Exposure of a subject to an immunogenic stimulus, such as an antigen (e.g., formulated as an antigenic composition or vaccine), elicits a primary immune response specific for the stimulus, that is, the exposure “primes” the immune response. A subsequent exposure, e.g., by immunization, to the stimulus can increase or “boost” the magnitude (or duration, or both) of the specific immune response. Thus, “boosting” a preexisting immune response by administering an antigenic composition increases the magnitude of an antigen-specific response, (e.g., by increasing antibody titer and/or affinity, by increasing the frequency of antigen specific B or T cells, by inducing maturation effector function, or a combination thereof).

In some or any aspects, the immunogen comprises a nucleic acid that encodes conserved FCoV1 CD8+ T cell epitope peptide. In some or any aspects, the immunogen comprises a nucleic acid that encodes a conserved FCoV2 CD8+ T cell epitope peptide. In some or any aspects, the immunogen comprises a nucleic acid that encodes a conserved SCoV2 CD8+ T cell epitope peptide

In some or any aspects, the conserved FCoV2 CD8+ T cell epitope peptide is an amino acid sequence set forth in any one of SEQ ID NOs: 16-18 and 44-67. In some or any aspects, the conserved SCoV2 CD8+ T cell epitope peptide is an amino acid sequence set forth in any one of SEQ ID NOs: 13-15 and 19-43.

In some or any aspects, the conserved CD8+ T cell epitope peptide is a FCoV2 nucleocapsid protein. In some or any aspects, the FCoV2 nucleocapsid protein is an amino acid sequence set forth in any one of SEQ ID NOs: 16-18.

In some or any aspects, the conserved CD8+ T cell epitope peptide is a SCoV2 nucleocapsid protein. In some or any aspects, the SCoV2 nucleocapsid protein is an amino acid sequence set forth in any one of SEQ ID NOs: 13-15.

In some or any aspects, the conserved CD8+ T cell epitope peptide is a SCoV2 polymerase enzyme epitope peptide. In some or any aspects, the SCoV2 polymerase enzyme epitope peptide is an amino acid sequence set forth in any one of SEQ ID NOs: 19-43.

In some or any aspects, the conserved CD8+ T cell epitope peptide is a FCoV2 polymerase enzyme epitope peptide. In some embodiments, the FCoV2 polymerase enzyme epitope peptide is an amino acid sequence set forth in any one of SEQ ID NOs: 44-67.

In some or any aspects, the conserved CD8+ T cell epitope peptide is a SCoV2 M-protease peptide. In some or any aspect, the SCoV2 M-protease peptide is an amino acid sequence set forth in any one of SEQ ID NOs: 68-75.

In some or any aspects, the conserved CD8+ T cell epitope peptide is a FCoV1 M-protease peptide. In some or any aspect, the FCoV1 M-protease peptide is an amino acid sequence set forth in any one of SEQ ID NOs: 76-83.

In some or any aspects, the adjuvant comprises a nucleic acid encoding an amino acid sequence composed of 2, 3, 4, 5, 6, 7, 8, 9, 10, 11, 12, 13, 14, 15, or 16 M-protease T cell epitope peptides set forth in SEQ ID NOs: 68-83, optionally conjugated together via a linker (e.g., a cathepsin S receptor linker, “KVSVR,” (SEQ ID NO: 84)). In some or any aspects, the adjuvant comprises a nucleic acid encoding an amino acid sequence composed of 2, 3, or 4 M-protease T cell epitope peptides set forth in SEQ ID NOs: 68-71, optionally conjugated together via a linker (e.g., a cathepsin S receptor linker, “KVSVR,” (SEQ ID NO: 84)). In some or any aspects, the adjuvant comprises a nucleic acid encoding an amino acid sequence set forth in SEQ ID NO: 89.

In some or any aspects, the immunogen comprises a nucleic acid encoding an amino acid sequence composed of 2, 3, or 4 M-protease T cell epitope peptides set forth in SEQ ID NOs: 72-75, optionally conjugated together via a linker (e.g., a cathepsin S receptor linker, “KVSVR,” (SEQ ID NO: 84)). In some or any aspects, the adjuvant comprises a nucleic acid encoding an amino acid sequence set forth in SEQ ID NO: 90.

In some or any aspects, the immunogen comprises a nucleic acid encoding an amino acid sequence composed of 2, 3, or 4 M-protease T cell epitope peptides set forth in SEQ ID NOs: 76-79, optionally conjugated together via a linker (e.g., a cathepsin S receptor linker, “KVSVR,” (SEQ ID NO: 84)). In some or any aspects, the adjuvant comprises a nucleic acid encoding an amino acid sequence set forth in SEQ ID NO: 91.

In some or any aspects, the immunogen comprises a nucleic acid encoding an amino acid sequence composed of 2, 3, or 4 M-protease T cell epitope peptides set forth in SEQ ID NOs: 80-83, optionally conjugated together via a linker (e.g., a cathepsin S receptor linker, “KVSVR,” (SEQ ID NO: 84)). In some or any aspects, the adjuvant comprises a nucleic acid encoding an amino acid sequence set forth in SEQ ID NO: 92.

In some or any aspects, the immunogen comprises a nucleic acid encoding an amino acid sequence comprising 2, 3, or 4 M-protease T cell epitopes and a Th1 peptide. Exemplary Th1 peptides include, but are not limited to, amino acid sequence set forth in SEQ ID NO: 85-88.

In some or any aspects, the immunogen comprises one or more nucleic acids encoding one or more M-protease amino acid sequences set forth in SEQ ID NO: 89, SEQ ID NO: 90, SEQ ID NO: 91 or SEQ ID NO: 92. In some or any aspects, the immunogen comprises one or more nucleic acids encoding one or more amino acid sequences set forth in SEQ ID NO: 89 and SEQ ID NO: 90. In some or any aspects, the immunogen comprises one or more nucleic acids encoding one or more amino acid sequences set forth in SEQ ID NO: 89 and SEQ ID NO: 91. In some or any aspects, the immunogen comprises one or more nucleic acids encoding one or more amino acid sequences set forth in SEQ ID NO: 89 and SEQ ID NO: 92. In some or any aspects, the immunogen comprises one or more nucleic acids encoding one or more amino acid sequences set forth in SEQ ID NO: 90 and SEQ ID NO: 91. In some or any aspects, the immunogen comprises amino acid sequences set forth in SEQ ID NO: 90 and SEQ ID NO: 92.

### Nucleic Acids, Vectors

Nucleic acid molecules comprising nucleotide sequences encoding the FCoV and SCoV2 RBD peptides (and optionally the conserved FCoV and/or SCoV2 CD8+ T cell epitope adjuvant peptides) are also contemplated. Optionally, the nucleic acid molecule(s) are inserted into a “vector.” As used herein, the term “vector” refers to any element, such as a plasmid, phage, transposon, cosmid, chromosome, virus, virus capsid, virion, etc., which is capable of transferring and/or transporting a nucleic acid to a host cell and/or that allows or facilitates the manipulation of a nucleic acid molecule. The disclosure contemplates naked or complexed nucleic acid molecules, as well as cloning vectors and expression vectors comprising any of the nucleic acid molecules described herein. The cloning or expression vector may be a viral vector or a non-viral vector.

In some or any aspects, the vector is a recombinant viral vector. In some embodiments, the recombinant viral vector is a recombinant adeno-associated vector (rAAV Vector).

An rAAV vector typically includes a serotype capsid protein that encapsulates a recombinant genome. The AAV genome typically includes functional 5′ and 3′ inverted terminal repeats sequences (ITR sequences). The ITR sequences are often flanked by exogenous nucleotide sequences that replace rep or cap genes found in wild-type AAVs. ITR sequences provide functional rescue, replication, and packaging to rAAVs. In some examples, the ITR sequences are from AAV2. In some or any aspects, the AAV is of the AAV1, AAV2, AAV4, AAV5, AAV6, AAV7, AAV8, AAV9, AAV10, AAV11, AAV 12, AAV13, AAVPHP.B, or AAVrh74 serotype.

In some or any aspects, nucleic acids encoding the FCoV (e.g., FCoV1 and/or FCoV2) and SCoV2 RBD peptides are present on separate vectors, although the nucleic acid sequences encoding all of the RBD peptides may be present on the same vector. In some or any aspects, nucleic acids encoding FCoV and/or SCoV2 CD8+ T cell epitope peptides are present on the same vector as the nucleic acids encoding the FCoV and SCoV2 RBD peptides. In some or any aspects, nucleic acids encoding the FCoV and SCoV2 RBD peptides are present on a separate (different) vector. In this regard, the disclosure provides a system comprising multiple different expression vectors comprising nucleic acids encoding different components of the composition disclosed herein.

Expression vectors typically comprise “expression regulatory elements,” which are generally a collection of promoter sequences, upstream regulatory regions, and transcriptional regulatory elements, which jointly drive replication, transcription and translation of coding region sequences.

### Formulations and Routes of Administration

In some or any aspects, the composition comprises a FCoV RBD peptide and a SCoV2 RBD peptide and a pharmaceutically acceptable carrier or excipient. Exemplary pharmaceutically acceptable excipients or carriers can include a buffer, such as Tris (trimethamine), phosphate (e.g. sodium phosphate), acetate, borate (e.g. sodium borate), citrate, glycine, histidine and succinate (e.g. sodium succinate), suitably sodium chloride, histidine, sodium phosphate or sodium succinate. The pharmaceutically acceptable excipient may include a salt, for example sodium chloride, potassium chloride or magnesium chloride. Optionally, the pharmaceutically acceptable excipient contains at least one component that stabilizes solubility and/or stability. Examples of solubilizing/stabilizing agents include detergents, for example, laurel sarcosine and/or polysorbate (e.g. Tween™80). Examples of stabilizing agents also include poloxamer (e.g. poloxamer 124, poloxamer 188, poloxamer 237, poloxamer 338 and poloxamer 407). The pharmaceutically acceptable excipient may include a non-ionic surfactant, for example polyoxyethylene sorbitan fatty acid esters, Polysorbate-80 (Tween™80), Polysorbate-60 (Tween™60), Polysorbate-40 (Tween™40) and Polysorbate-20 (Tween™20), or polyoxyethylene alkyl ethers (suitably polysorbate-80). Alternative solubilizing/stabilizing agents include arginine, and glass forming polyols (such as sucrose, trehalose and the like). The pharmaceutically excipient may be a preservative, for example phenol, 2-phenoxyethanol, or thiomersal. Other pharmaceutically acceptable excipients include sugars (e.g. lactose, sucrose), and proteins (e.g. gelatine and albumin). Pharmaceutically acceptable carriers include water, saline solutions, aqueous dextrose and glycerol solutions

In some or any aspects, the composition is formulated in a nanoparticle, a liposome, a microparticle, a microsphere, a nanosphere, a unilamellar vesicle, a multilamellar vesicle, or a virus-like particle (VLP).

In some or any aspects, the composition is formulated in a nanoparticle (e.g., a lipid nanoparticle). Optionally, the nanoparticle has a mean diameter of 50-200 nm. In some or any aspects, the lipid nanoparticle comprises DLin-MC3-DMA ionizable lipid and four structural lipids: 1,2-dipalmitoyl-sn-glycero-3-phosphocholine (DPPC), 1,2-dioleoyl-sn-glycero-3-phosphoethanolamine (DOPE), and 1,2-dimyristoyl-sn-glycero-3-methoxypolyethyleneglycol 2000 (DMG-Peg), and cholesterol. In some or any aspects, the nanoparticle is specific for a T cell. In some or any aspects, the nanoparticle is specific for a dendritic cell.

The compositions described herein can be administered by any route, including a systemic or mucosal route. Exemplary administrations may include injection via the intramuscular (IM), intraperitoneal (IP), intradermal (ID) or subcutaneous (SC) routes; or via mucosal administration to the oral/alimentary, respiratory, genitourinary tracts. Following an initial administration, subjects may receive one or several booster administrations.

### Methods of Use

The disclosure provides a method of treating or preventing a coronavirus infection, the method comprising administering an composition described herein to a subject in need thereof. The disclosure further provides a method of stimulating an immune response in a subject in need thereof. The method comprises administering to the subject an effective amount of the composition, thereby stimulating an immune response in the subject. In some or any aspects, administering the composition induces a B cell response against (i) SARS-CoV-2, (ii) FCoV1, and (iii) FCoV2. Alternative or in addition, administering the composition optionally induces a CD8+ and/or CD4+ T cell response against (i) SARS-CoV-2, (ii) FCoV1, and (iii) FCoV2.

In some or any aspects, the method comprises administering a first composition comprising a nucleic acid encoding a FCoV RBD peptide and a SCoV2 RBD peptide described herein and a second composition comprising one or more nucleic acids encoding one or more FCoV and/or SCoV2 CD8+ T cell epitope peptides.

In some or any aspects, the first composition comprises a nucleic acid encoding FCoV serotype 1 (FCoV1) RBD peptide. In some or any aspects, the composition comprises a nucleic acid encoding a FCoV1 RBD peptide comprising an amino acid sequence that is at least 70% identical to the amino acid sequence set forth in SEQ ID NO: 5. In some or any aspects, the composition comprises a nucleic acid encoding a FCoV1 RBD peptide comprising an amino acid sequence that is at least 70% (e.g., at least 80%, 85%, 90%, 91%, 92%, 93%, 94%, 95%, 96%, 97%, 98%, 99% or more) identical to the amino acid sequence set forth in SEQ ID NO: 5. In some or any aspects, the composition comprises a nucleic acid encoding a FCoV1 RBD peptide comprising the amino acid sequence set forth in SEQ ID NO: 5.

In some or any aspects, the first composition comprises a nucleic acid encoding a FCoV serotype 2 (FCoV2) RBD peptide. In some or any aspects, the first composition comprises a nucleic acid encoding a FCoV2 RBD peptide comprising an amino acid sequence that is at least 70% identical to the amino acid sequence set forth in SEQ ID NO: 7. In some or any aspects, the first composition comprises a nucleic acid encoding a FCoV2 RBD peptide comprising an amino acid sequence that is at least 70% (e.g., at least 80%, 85%, 90%, 91%, 92%, 93%, 94%, 95%, 96%, 97%, 98%, 99% or more) identical to the amino acid sequence set forth in SEQ ID NO: 7. In some or any aspects, the first composition comprises a nucleic acid encoding a FCoV2 RBD peptide comprising the amino acid sequence set forth in SEQ ID NO: 7.

In some or any aspects, the first composition comprises a nucleic acid encoding a SCoV2 RBD peptide comprising an amino acid sequence that is at least 70% identical to the amino acid sequence set forth in SEQ ID NO: 1 (UF-RBD). In some or any aspects, the first composition comprises a nucleic acid encoding a SCoV2 RBD peptide comprising an amino acid sequence that is at least 70% (e.g., at least 80%, 85%, 90%, 91%, 92%, 93%, 94%, 95%, 96%, 97%, 98%, 99% or more) identical to the amino acid sequence set forth in SEQ ID NO: 1. In some or any aspects, the first composition comprises a nucleic acid encoding a SCoV2 RBD peptide comprising the amino acid sequence set forth in SEQ ID NO: 1.

In some or any aspects, the first composition comprises a nucleic acid encoding a SCoV2 RBD peptide comprising an amino acid sequence that is at least 70% identical to the amino acid sequence set forth in SEQ ID NO: 3 (MB-RBD). In some or any aspects, the first composition comprises a nucleic acid encoding a SCoV2 RBD peptide comprising an amino acid sequence that is at least 70% (e.g., at least 80%, 85%, 90%, 91%, 92%, 93%, 94%, 95%, 96%, 97%, 98%, 99% or more) identical to the amino acid sequence set forth in SEQ ID NO: 3. In some or any aspects, the first composition comprises a nucleic acid encoding a SCoV2 RBD peptide comprising the amino acid sequence set forth in SEQ ID NO: 3.

In some embodiments, the first composition comprises a nucleic acid encoding a SCoV2 RBD peptide comprising an amino acid sequence that is at least 70% identical to the amino acid sequence set forth in SEQ ID NO: 9. In some or any aspects, the first composition comprises a nucleic acid encoding a SCoV2 RBD peptide comprising an amino acid sequence that is at least 70% (e.g., at least 80%, 85%, 90%, 91%, 92%, 93%, 94%, 95%, 96%, 97%, 98%, 99% or more) identical to the amino acid sequence set forth in SEQ ID NO: 9. In some or any aspects, the first composition comprises a nucleic acid encoding a SCoV2 RBD peptide comprising the amino acid sequence set forth in SEQ ID NO: 9.

In some or any aspects, the first composition comprises a nucleic acid encoding a SCoV2 RBD peptide comprising an amino acid sequence that is at least 70% identical to the amino acid sequence set forth in SEQ ID NO: 10. In some or any aspects, the first composition comprises a nucleic acid encoding a SCoV2 RBD peptide comprising an amino acid sequence that is at least 70% (e.g., at least 80%, 85%, 90%, 91%, 92%, 93%, 94%, 95%, 96%, 97%, 98%, 99% or more) identical to the amino acid sequence set forth in SEQ ID NO: 10. In some or any aspects, the first composition comprises a nucleic acid encoding a SCoV2 RBD peptide comprising the amino acid sequence set forth in SEQ ID NO: 10.

In some or any aspects, the first composition comprises a nucleic acid encoding a FCoV1 RBD domain peptide comprising an amino acid sequence that is at least 70% (e.g., at least 80%, 85%, 90%, 91%, 92%, 93%, 94%, 95%, 96%, 97%, 98%, 99% or more) identical to the amino acid sequence set forth in SEQ ID NO: 5 and a SCoV2 RBD peptide comprising an amino acid sequence that is at least 70% (e.g., at least 80%, 85%, 90%, 91%, 92%, 93%, 94%, 95%, 96%, 97%, 98%, 99% or more) identical to the amino acid sequence set forth in SEQ ID NO:1. In some or any aspects, the composition comprises a nucleic acid encoding a FCoV1 RBD peptide comprising the amino acid sequence set forth in SEQ ID NO: 5 and a SCoV2 RBD peptide comprising the amino acid sequence set forth in SEQ ID NO: 1.

In some or any aspects, the first composition comprises a nucleic acid encoding a FCoV1 RBD domain peptide comprising an amino acid sequence that is at least 70% (e.g., at least 80%, 85%, 90%, 91%, 92%, 93%, 94%, 95%, 96%, 97%, 98%, 99% or more) identical to the amino acid sequence set forth in SEQ ID NO: 5 and a SCoV2 RBD peptide comprising an amino acid sequence that is at least 70% (e.g., at least 80%, 85%, 90%, 91%, 92%, 93%, 94%, 95%, 96%, 97%, 98%, 99% or more) identical to the amino acid sequence set forth in SEQ ID NO: 3. In some or any aspects, the first composition comprises a nucleic acid encoding a FCoV1 RBD peptide comprising the amino acid sequence set forth in SEQ ID NO: 5 and a SCoV2 RBD peptide comprising the amino acid sequence set forth in SEQ ID NO: 3.

In some or any aspects, the first composition comprises a nucleic acid encoding a FCoV1 RBD domain peptide comprising an amino acid sequence that is at least 70% (e.g., at least 80%, 85%, 90%, 91%, 92%, 93%, 94%, 95%, 96%, 97%, 98%, 99% or more) identical to the amino acid sequence set forth in SEQ ID NO: 5 and a SCoV2 RBD peptide comprising an amino acid sequence that is at least 70% (e.g., at least 80%, 85%, 90%, 91%, 92%, 93%, 94%, 95%, 96%, 97%, 98%, 99% or more) identical to the amino acid sequence set forth in SEQ ID NO. 9. In some or any aspects, the first composition comprises a nucleic acid encoding a FCoV1 RBD peptide comprising the amino acid sequence set forth in SEQ ID NO: 5 and a SCoV2 RBD peptide comprising the amino acid sequence set forth in SEQ ID NO: 9.

In some or any aspects, the first composition comprises a nucleic acid encoding a FCoV1 RBD domain peptide comprising an amino acid sequence that is at least 70% (e.g., at least 80%, 85%, 90%, 91%, 92%, 93%, 94%, 95%, 96%, 97%, 98%, 99% or more) identical to the amino acid sequence set forth in SEQ ID NO: 5 and a SCoV2 RBD peptide comprising an amino acid sequence that is at least 70% (e.g., at least 80%, 85%, 90%, 91%, 92%, 93%, 94%, 95%, 96%, 97%, 98%, 99% or more) identical to the amino acid sequence set forth in SEQ ID NO: 10. In some or any aspects, the first composition comprises a nucleic acid encoding a FCoV1 RBD peptide comprising the amino acid sequence set forth in SEQ ID NO: 5 and a SCoV2 RBD peptide comprising the amino acid sequence set forth in SEQ ID NO: 10.

In some or any aspects, the first composition comprises a nucleic acid encoding a FCoV2 RBD peptide comprising an amino acid sequence that is at least 70% (e.g., at least 80%, 85%, 90%, 91%, 92%, 93%, 94%, 95%, 96%, 97%, 98%, 99% or more) identical to the amino acid sequence set forth in SEQ ID NO: 7 and a SCoV2 RBD peptide comprising an amino acid sequence that is at least 70% (e.g., at least 80%, 85%, 90%, 91%, 92%, 93%, 94%, 95%, 96%, 97%, 98%, 99% or more) identical to the amino acid sequence set forth in SEQ ID NO: 1. In some or any aspects, the first composition comprises a nucleic acid encoding a FCoV2 RBD peptide comprising the amino acid sequence set forth in SEQ ID NO: 7 and a SCoV2 RBD peptide comprising the amino acid sequence set forth in SEQ ID NO: 1.

In some or any aspects, the first composition comprises a nucleic acid encoding a FCoV2 RBD peptide comprising an amino acid sequence that is at least 70% (e.g., at least 80%, 85%, 90%, 91%, 92%, 93%, 94%, 95%, 96%, 97%, 98%, 99% or more) identical to the amino acid sequence set forth in SEQ ID NO: 7 and a SCoV2 RBD peptide comprising an amino acid sequence that is at least 70% (e.g., at least 80%, 85%, 90%, 91%, 92%, 93%, 94%, 95%, 96%, 97%, 98%, 99% or more) identical to the amino acid sequence set forth in SEQ ID NO: 3. In some or any aspects, the first composition comprises a nucleic acid encoding a FCoV2 RBD peptide comprising the amino acid sequence set forth in SEQ ID NO: 7 and a SCoV2 RBD peptide comprising the amino acid sequence set forth in SEQ ID NO: 3.

In some or any aspects, the first composition comprises a nucleic acid encoding a FCoV2 RBD peptide comprising an amino acid sequence that is at least 70% (e.g., at least 80%, 85%, 90%, 91%, 92%, 93%, 94%, 95%, 96%, 97%, 98%, 99% or more) identical to the amino acid sequence set forth in SEQ ID NO: 7 and a SCoV2 RBD peptide comprising an amino acid sequence that is at least 70% (e.g., at least 80%, 85%, 90%, 91%, 92%, 93%, 94%, 95%, 96%, 97%, 98%, 99% or more) identical to the amino acid sequence set forth in SEQ ID NO: 9. In some or any aspects, the first composition comprises a nucleic acid encoding a FCoV2 RBD peptide comprising the amino acid sequence set forth in SEQ ID NO: 7 and a SCoV2 RBD peptide comprising the amino acid sequence set forth in SEQ ID NO: 9.

In some or any aspects, the first composition comprises a nucleic acid encoding a FCoV2 RBD peptide comprising an amino acid sequence that is at least 70% (e.g., at least 80%, 85%, 90%, 91%, 92%, 93%, 94%, 95%, 96%, 97%, 98%, 99% or more) identical to the amino acid sequence set forth in SEQ ID NO: 7 and a SCoV2 RBD peptide comprising an amino acid sequence that is at least 70% (e.g., at least 80%, 85%, 90%, 91%, 92%, 93%, 94%, 95%, 96%, 97%, 98%, 99% or more) identical to the amino acid sequence set forth in SEQ ID NO: 10. In some or any aspects, the first composition comprises a nucleic acid encoding a FCoV2 RBD peptide comprising the amino acid sequence set forth in SEQ ID NO: 7 and a SCoV2 RBD peptide comprising the amino acid sequence set forth in SEQ ID NO: 10.

In some or any aspects, the first composition comprises a nucleic acid encoding a FCoV1 RBD peptide comprising an amino acid sequence that is at least 70% (e.g., at least 80%, 85%, 90%, 91%, 92%, 93%, 94%, 95%, 96%, 97%, 98%, 99% or more) identical to the amino acid sequence set forth in SEQ ID NO: 5, a FCoV2 RBD peptide comprising an amino acid sequence that is at least 70% (e.g., at least 80%, 85%, 90%, 91%, 92%, 93%, 94%, 95%, 96%, 97%, 98%, 99% or more) identical to the amino acid sequence set forth in SEQ ID NO: 7, and a SCoV2 RBD peptide comprising an amino acid sequence that is at least 70% (e.g., at least 80%, 85%, 90%, 91%, 92%, 93%, 94%, 95%, 96%, 97%, 98%, 99% or more) identical to the amino acid sequence set forth in SEQ ID NO: 1, SEQ ID NO: 3, SEQ ID NO: 9, or SEQ ID NO: 10.

In some or any aspects, the first composition comprises a nucleic acid encoding a FCoV1 RBD peptide comprising the amino acid sequence set forth in SEQ ID NO: 5, a FCoV2 RBD peptide comprising the amino acid sequence set forth in SEQ ID NO: 7, and a SCoV2 peptide comprising the amino acid sequence set forth in SEQ ID NO: 1. In some or any aspects, the first composition comprises a nucleic acid encoding a FCoV1 RBD peptide comprising the amino acid sequence set forth in SEQ ID NO: 5, a FCoV2 RBD peptide comprising the amino acid sequence set forth in SEQ ID NO: 7, and a SCoV2 peptide comprising the amino acid sequence set forth in SEQ ID NO: 3. In some or any aspects, the first composition comprises a nucleic acid encoding a FCoV1 RBD peptide comprising the amino acid sequence set forth in SEQ ID NO: 5, a FCoV2 RBD peptide comprising the amino acid sequence set forth in SEQ ID NO: 7, and a SCoV2 peptide comprising the amino acid sequence set forth in SEQ ID NO: 9. In some or any aspects, the first composition comprises a nucleic acid encoding a FCoV1 RBD peptide comprising the amino acid sequence set forth in SEQ ID NO: 5, a FCoV2 RBD peptide comprising the amino acid sequence set forth in SEQ ID NO: 7, and a SCoV2 peptide comprising the amino acid sequence set forth in SEQ ID NO: 10.

In some or any aspects, the first composition optionally further comprises a nucleic acid encoding a canine coronavirus serotype 1 (CCoV1) RBD peptide or a CCoV serotype 2 (CCoV2) RBD peptide. In some or any aspects, the CCoV1 RBD peptide comprises an amino acid sequence that is at least 70% (e.g., at least 80%, 85%, 90%, 91%, 92%, 93%, 94%, 95%, 96%, 97%, 98%, 99% or more) identical to the amino acid sequence set forth in SEQ ID NO: 11. In some or any aspects, the CCoV1 RBD peptide comprises the amino acid sequence set forth in SEQ ID NO: 11. In some or any aspects, the CCoV2 RBD peptide comprises an amino acid sequence that is at least 70% (e.g., at least 80%, 85%, 90%, 91%, 92%, 93%, 94%, 95%, 96%, 97%, 98%, 99% or more) identical to the amino acid sequence set forth in SEQ ID NO: 12. In some or any aspects, the CCoV2 RBP comprises the amino acid sequence set forth in SEQ ID NO: 12.

In some or any aspects, the first composition comprises an amino acid sequence that is at least 70% (e.g., at least 80%, 85%, 90%, 91%, 92%, 93%, 94%, 95%, 96%, 97%, 98%, 99% or more) identical to the amino acid sequence set forth in SEQ ID NO: 93. In some or any aspects, the first composition comprises an amino acid sequence set forth in SEQ ID NO: 93. In some or any aspects, the first composition comprises an amino acid sequence that is at least 70% (e.g., at least 80%, 85%, 90%, 91%, 92%, 93%, 94%, 95%, 96%, 97%, 98%, 99% or more) identical to the amino acid sequence set forth in SEQ ID NO: 94. In some or any aspects, the first composition comprises an amino acid sequence set forth in SEQ ID NO: 94. In some or any aspects, the first composition comprises an amino acid sequence that is at least 70% (e.g., at least 80%, 85%, 90%, 91%, 92%, 93%, 94%, 95%, 96%, 97%, 98%, 99% or more) identical to the amino acid sequence set forth in SEQ ID NO: 95. In some or any aspects, the first composition comprises an amino acid sequence set forth in SEQ ID NO: 95.

In some or any aspects, the second composition comprises a conserved FCoV2 CD8+ T cell epitope peptide comprising a nucleic acid that encodes an amino acid sequence set forth in any one of SEQ ID NOs: 16-18 and 44-67, a SCoV2 CD8+ T cell epitope peptide comprising an amino acid sequence set forth in any one of SEQ ID NOs: 13-15 and 19-43, or combinations thereof.

In some or any aspects, the second composition comprises a nucleic acid encoding a SCoV2 M-protease peptide comprising amino acid sequence set forth in any one of SEQ ID NOs: 68-75, a FCoV1 M-protease peptide is an amino acid sequence set forth in any one of SEQ ID NOs: 76-83, or combinations thereof.

In some or any aspects, the second composition comprises a nucleic acid encoding an amino acid sequence composed of 2, 3, 4, 5, 6, 7, 8, 9, 10, 11, 12, 13, 14, 15, or 16 M-protease T cell epitope peptides set forth in SEQ ID NOs: 68-83, optionally conjugated together via a linker (e.g., a cathepsin S receptor linker, “KVSVR,” (SEQ ID NO: 84)). In some or any aspects, the second composition comprises a nucleic acid encoding an amino acid sequence composed of 2, 3, or 4 M-protease T cell epitope peptides set forth in SEQ ID NOs: 68-71, optionally conjugated together via a linker (e.g., a cathepsin S receptor linker, “KVSVR,” (SEQ ID NO: 84)). In some or any aspects, the second composition comprises a nucleic acid encoding the amino acid sequence set forth in SEQ ID NO: 89.

In some or any aspects, the second composition comprises a nucleic acid encoding an amino acid sequence composed of 2, 3, or 4 M-protease T cell epitope peptides set forth in SEQ ID NOs: 72-75, optionally conjugated together via a linker (e.g., a cathepsin S receptor linker, “KVSVR,” (SEQ ID NO: 84)). In some or any aspects, the second composition comprises a nucleic acid sequence encoding the amino acid sequence set forth in SEQ ID NO: 90.

In some or any aspects, the second composition comprises a nucleic acid encoding an amino acid sequence composed of 2, 3, or 4 M-protease T cell epitope peptides set forth in SEQ ID NOs: 76-79, optionally conjugated together via a linker (e.g., a cathepsin S receptor linker, “KVSVR,” (SEQ ID NO: 84)). In some or any aspects, the second composition comprises a nucleic acid encoding the amino acid sequence set forth in SEQ ID NO: 91.

In some or any aspects, the second composition comprises a nucleic acid encoding an amino acid sequence composed of 2, 3, or 4 M-protease T cell epitope peptides set forth in SEQ ID NOs: 80-83, optionally conjugated together via a linker (e.g., a cathepsin S receptor linker, “KVSVR,” (SEQ ID NO: 84)). In some or any aspects, the second composition comprises a nucleic acid encoding the amino acid sequence set forth in SEQ ID NO: 92.

In some or any aspects, the nucleic acid encoding an amino acid sequence comprising 2, 3, or 4 M-protease T cell epitope peptides further comprises a Th2 peptide. Exemplary Th2 peptides include, but are not limited to, amino acid sequences set forth in SEQ ID NO: 85-88.

In some or any aspects, the second composition comprises one or more nucleic acids encoding M-protease amino acid sequences set forth in SEQ ID NO: 89, SEQ ID NO: 90, SEQ ID NO: 91 or SEQ ID NO: 92. In some or any aspects, the second composition comprises one or more nucleic acids encoding M-protease amino acid sequences set forth in SEQ ID NO: 89 and SEQ ID NO: 90. In some or any aspects, the second composition comprises one or more nucleic acids encoding amino acid sequences set forth in SEQ ID NO: 89 and SEQ ID NO: 91. In some or any aspects, the second composition comprises amino acid sequences set forth in SEQ ID NO: 89 and SEQ ID NO: 92. In some or any aspects, the second compositions comprises one or more nucleic acids encoding M-protease amino acid sequences set forth in SEQ ID NO: 90 and SEQ ID NO: 91. In some or any aspects, the second composition comprises one or more nucleic acids encoding M-protease amino acid sequences set forth in SEQ ID NO: 90 and SEQ ID NO: 92.

In some or any aspects, the first composition and second composition are formulated in a lipid nanoparticle. In some or any aspects, the second composition is formulation is a different (separate) lipid nanoparticle. In some or any aspects, the lipid nanoparticle is T cell specific. In some or any aspects, the lipid nanoparticle is specific for a dendritic cell.

The terms “subject”, “subject in need”, and “individual” refer to an animal, in particular vertebrates, such as mammals. The subject is, in various aspects, a cat, a dog, a hamster, or a human, although other animals also are contemplated. The subject may not be diagnosed with coronavirus infection and/or experiencing symptoms; alternatively, the subject may be suffering from a coronavirus infection and/or suffering from symptoms of a coronavirus infection and/or diagnosed with a coronavirus infection. In some or any aspects, the subject has been exposed to a coronavirus (e.g., the subject has come in contact with a person or an animal that is infected with a coronavirus). Alternatively, the subject may be at risk of being exposed to a coronavirus.

The term “treatment” or “treat” refers to an intervention made in response to a disease, disorder or physiological condition (e.g., a coronavirus infection or symptoms associated with the infection) manifested by the subject. Treatment does not require the complete curing of a disorder; “treatment” encompasses reduction of symptoms associated with a coronavirus infection. Exemplary symptoms of a coronavirus infection include, but are not limited to, fever, cough, tiredness, a loss of taste or smell, shortness of breath or difficulty breathing, muscle aches, chills, sore throat, runny nose, headache, chest pain, pink eye (conjunctivitis), nausea, vomiting, diarrhea, rash, pneumonia and acute respiratory distress syndrome.

As used herein, the term “prevention” refer to a reduced likelihood that a subject will be susceptible to a coronavirus infection, or less susceptible to symptoms associated with a coronavirus infection. Rather, it denotes that the likelihood of the occurrence of the event has been reduced by the instant method.

The phrase “effective amount” as used herein means that amount of the composition disclosed herein which is effective for inducing an immune response in the subject. In some embodiments, an effective amount is necessary to inhibit coronavirus replication or to measurably alleviate outward symptoms of the viral infection. In some embodiments, a therapeutically effective amount is an amount that prevents one or more signs or symptoms that can be caused by a coronavirus infection.

In some embodiments, the determination of an effective amount of the vaccine composition can be measured by measuring the titer of antibodies produced against a coronavirus. Methods of determining antibody titers are described in the example section of the present disclosure (see, for example, Examples 1, 4, and 5).

### EXAMPLES

### Materials and Methods

Animal populations: The studies used laboratory cats that were bred and cared for under the University of Florida (UF) IACUC protocols 201801838, 201101838, 201401838, 201701838, and 202001838, for over a decade. The specific pathogen-free (SPF) toms were inbred from SPF cats initially derived from intact female (Vendor—Harlan Sprague Dawley, Inc., Indianapolis, IN) and male SPF cats (Vendor—Cedar River Laboratories, Mason City, IA). Four queens were donated by the University of Georgia (UGA) in fall 2019, which were purchased by UGA from Liberty Research Inc (Waverly, NY). Juvenile cats were generated by accidental mating of the donated queens with the three SPF toms at a UF laboratory, with the goal to outbreed the inbred SPF colony for higher fertility with larger litter size. The eight group-housed laboratory cats were initially purchased from Liberty Research, Inc. However, their animal code did not show the same lineages as the UGA queens and were considered different cat lineages (i.e., different origin), unrelated to both UGA queens and UF toms. All cats purchased from Liberty Research, Inc. were vaccinated against rabies, using the RABVAC3 vaccine, and against feline panleukopenia (distemper), calici, and rhinotracheitis (herpes) viruses, along with hemorrhagic feline calicivirus strain, using the Fel-O-Vax PCT+Calicivax vaccine.

TABLE 1
the study population

| SPF Inbred Toms (UF) | Queens (UGA) | Juvenile cats |
|---|---|---|
| 5HQT1 | UGAQ1 | Zero - (Mating unsuccessful) |
| HOGT3 | UGAQ2 | Zero - (Mating unsuccessful) |
| 5HQT1 | UGAQ3 | Y2B |
| HOJT2 | UGAQ4 | D4A, D4D, D4F |

| | |
|---|---|
| Eight Separate | G-1, G-2, G-3, G-4, G-5, |
| Laboratory Cats: | G-6, G-7, and G-8 |

### SARS-CoV-2 RBD Peptides

Two different versions of SCoV2, University of Florida-RBD (UF-RBD, SEQ ID NO. 1) and MassBiologic-RBD (MB-RBD, SEQ ID NO: 3) were used. The UF-RBD was produced using Harvard Wuhan RBD plasmid²¹ and expressed in EXPI293F cells. An SCoV2 RBD set forth in SEQ ID NO: 9 and SEQ ID NO: 10 was also used.

Feline cell lines: Crandell feline kidney (CrFK) fibroblast, Felis catus 9 (Fc9), and felis catus whole fetus-4 (Fcwf-4) cells were provided by Dr. N. C. Pederson of the University of California, Davis. All of these feline cell lines were maintained on Eagle MEM media with 1.5 g/L sodium bicarbonate, NEAA, L-glutamine, and sodium pyruvate supplemented with 10% fetal bovine serum (FBS) with 50 μg/mL gentamycin. These cells were maintained at 37° C. with 5% CO2 and passaged every 2-3 days using 0.25% Trypsin-EDTA (Gibco-Life Technologies, Grand Island, NY). In order to prevent changes in growth patterns, these cells were culture amplified and then cryopreserved in liquid nitrogen tanks in multiple vials.

Production and partial purification of FCoV2 whole-virus: The CrFK cells were infected with FCoV2 WSU79-1146, purchased from ATCC to produce the stock of crude FCoV2 inoculum for in vitro infection studies, and partially purified FCoV2 whole-virus (WV) stock for ELISA and immunoblots. The infected culture fluids directly from the culture flask (175 cm) were pooled and clarified free of cell debris by low-speed centrifugation at 2800-3000 rpm for 45 min at 5° C. In addition, the freeze-thawed culture fluids from the frozen flasks with residual media were pooled and then clarified free of cell debris by low-speed centrifugation at 2800-3000 rpm for 45 min at 5° C. They were then combined with a portion of the clarified culture fluid from above, at one part clarified direct culture fluid to two parts of clarified cell-debris fluid. Both direct and combined clarified fluids were partially purified by the following three methods. In the first method, the clarified fluid was purified by ultracentrifugation at 16,000 rpm for 2 h at 5° C. to obtain pooled virus pellets. The pellets then underwent multiple washes with PBS, while being concentrated by Vivaspin 20 Centrifugal Concentrators (Satorius, Gottingen, Germany) with PES membrane of 100k molecular weight cut off (MWCO). In the second method, the combined clarified fluid was directly concentrated by Vivaspin 20 Centrifugal Concentrators by 10-50-fold, while the concentrated virus fluid underwent additional 3-5 washes with PBS using the Centrifugal Concentrator. The multiple washes with PBS using the Centrifugal Concentrator 100k were performed until the red color of the phenol red was not present in the preparation from the first method. This method resulted in a FCoV2-WV stock preparation with no detectable residual BSA (67 kDa), but the virus titer (670 μg/mL) was lower than the second method and had undetectable 180 kDa S glycoprotein. The second method resulted in a preparation with an extremely light pink color from the phenol red of the media, but a reasonably high FCoV2 load (1.0 mg/mL) with detectable levels of 180 kDa S glycoprotein. This method was used the most for immunoblot production. The last method consisted of direct concentration of direct-clarified fluid using multiple Centrifugal Concentrators with minimal PBS washes. As a result, this preparation retained the phenol red color and about 5% residual BSA from FBS. This method provided a virus preparation at a high concentration (150 mg/mL), needed for use in screening a large number of cat sera with FCoV2-WV ELISA.

Transfection and expression of Expi293F Cells with SCoV2, FCoV1, and FCoV2 RBD plasmids and purification of RBD proteins: The plasmids pVRC containing RBD constructs were transiently transfected into Expi293F cells using the ExpiFectamine 293 Transfection Kit (Thermo Fisher, Waltham, MA). Briefly, the cell density was adjusted to 3×10⁶ cells/mL in a final volume of 100 mL of Expi293 expression media and allowed to grow 24 h to reach a final density of 5.5×10⁶ cells/mL. The plasmid DNA (1 μg/mL) and ExpiFectamine 293 reagent were individually diluted with Opti-MEM Reduced Serum Medium (Thermo Fisher, Waltham, MA), incubated 5 minutes at room temperature, and then mixed together. The ExpiFectamine 293/plasmid DNA mixture was incubated at room temperature for 20 minutes and mixed with Expi293F cells. The cells were incubated on an orbital shaker in a 37° C. incubator with 8% CO₂. After 24 h of incubation, transfection enhancers-1 and 2 were added and incubated for three days. Then, the cell culture was centrifuged at 1800×g for 30 min to collect the supernatant for protein purification. The culture supernatant was concentrated to a final volume of 5 mL using a Macrosep Omega Advance Centrifugal Device with a cutoff of 10 kDa (PALL Laboratory, Port Washington, NY). The concentrated supernatant was passed through equilibrated TALON Metal Affinity Resin (Takara Bio Inc, Shiga, Japan). The column was washed with 10 volume of PBS containing NaCl (300 mM) and imidazole (20 mM) to remove all contaminants. Subsequently, the protein was eluted from the column using PBS containing 250 mM imidazole. The eluted fractions were concentrated using a Nanosep Advance Centrifugal Device with 10K Omega (PALL Laboratory). Estimation of protein purity and quantity were achieved with SDS-PAGE and a Pierce BCA Protein Assay Kit (Thermo Fisher Scientific, Rockford, IL), respectively.

### Enzyme-Linked Immunosorbent Assay (ELISA)

FCoV whole-virus and SCoV2 RBD ELISAs with overnight serum incubation: ELISA plate (Costa, Corning, NY, USA) wells were coated with 100 μL of 100 μg/mL FCoV2 whole virus antigen or 100 μg/mL SCoV2 RBD or FCoV RBD antigen in sodium bicarbonate ELISA coating buffer, pH 9.5 (BioLegend, San Diego, CA) and incubated overnight at 4° C. The next day, the plates were washed three times with phosphate-buffered saline tween (PBST). Non-specific binding sites were blocked with 100 μL per well of blocking solution (5% skimmed milk in sterile PBST-0.5% Tween-20) for 1 h at 37° C. After washing with PBST three times, 50 μL of cat sera was diluted to 1:100 with the blocking solution and incubated at room temperature overnight. After washing with PBST three times using the BioTek ELx-405 plate washer, horseradish peroxidase-conjugated goat anti-cat IgG (HRP-anti-cat-IgG) diluted 1:4,000 (SouthernBioTech, Birmingham, AL) in PBST was added and incubated at room temperature (RT) for 2 h. After washing, 100 μL of 3,3,5,5-tetramethylbenzidine (TMB) substrate solution (BioLegend) was added to the wells and incubated at RT for 15 min, the reaction was stopped by adding 100 μL of 1 N HCl in sterile water as stop solution. The ELISA titer was measured at OD450 using BioTek's Synergy HTX Multi-Mode Microplate Reader (BioTek, Winooski, VT, USA).

Stringent FCoV whole-virus and SCoV2 RBD ELISAs with one hour serum incubation: To ensure that the serum ELISA reactivity was specific to the FCoV2 whole-virus, FCoV-RBD or SCoV2-RBD antigen, sera from all four UGA queens and three toms were incubated individually at the same dilution for only 1 h, instead of overnight. PBS was used instead of bicarbonate buffer for coating of the antigen on the ELISA plates. In addition, a bovine serum albumin (BSA) antigen control was included since veterinary vaccines used at the time the UGA queens were contained contaminating BSA, most likely from the cell cultures used during the manufacture of the viral vaccines. The BSA control was also important because the FCoV2 whole-virus preparation contained about 5% BSA, whereas the RBDs were highly purified and devoid of BSA.

Immunoblot analysis using FCoV2-WV, SCoV2 UF-RBD, FCoV1 RBD, and FCoV2 RBD as antigen substrate: The purified proteins were analyzed by SDS-PAGE and western blot, also called immunoblot. Briefly, the proteins (100 μg) were boiled at 95° C. for 5 min in a sample buffer (bromophenol blue, glycerol, reducing agent β-mercaptoethanol, and SDS in Tris-HCL buffer). They were loaded evenly into a single, 7-cm wide well of the stacking gel, with one 4-mm wide well at the end for pre-stained marker, and separated by 10% Tris-HCL gel with 30% or 40% acrylamide/bis. The proteins were transferred to nitrocellulose membrane for immunoblot analysis. Each nitrocellulose blot was cut vertically into 26 3.2-mm width strips using a Novex Model NZ-1CIS membrane strip cutter (Novel Experimental Technology, San Diego, CA). Each mini-gel provided 21-22 strips of the antigen (RBD or FCoV2 whole-virus), with two strips on one end containing the Precision Plus Protein All Blue Pre-stained Standards (Bio-Rad). Two strips on the other end were discarded for lack of or uneven antigen presence. Each immunoblot strip was incubated individually with FCoV-infected cat serum or plasma at a dilution of 1:50 or 1:100 in a blocking buffer containing 5% non-fat dry milk in PBST and incubated overnight at RT on a rocker. After three washes, the strips were then incubated with alkaline phosphatase (AP)-conjugated goat anti-cat IgG (1:1,000) (SouthernBiotech) for 2 h at RT. Subsequently after three washes, the reactive bands were visualized with freshly mixed AP substrate reagents from AP-Conjugate Substrate Kit (Bio-Rad). Upon drying, the strips were aligned and scanned at a designated brightness and contrast.

FCoV2 neutralizing antibody (NAb) assay and RBD blocking assay against FCoV2: The Fc9 cells were used for both FCoV2 neutralizing antibody studies and FCoV2/SCoV2 RBD blocking studies against live FCoV2 infection. The FCoV2 NAb assay was a modification of FCoV2 NAb assay described previously.²³ The modification consisted of a 96-well round-bottom microculture plate system where the diluted FCoV2 preparation (EMEM culture media with 5% heat-inactivated FBS) at 2 TCID₅₀ were incubated with equal volume of serially three-fold diluted cat serum (EMEM culture media at 5% heat-inactivated FBS). The plates were incubated at 37° C. in a 5% CO₂ incubator for 45 min. Note that the first row of the 12 wells were not used in order to prevent the drying effect on the 12 edge wells with the most concentrated serum dilution. Thus, the most concentrated serum dilutions of 1:6 with the virus preparation (i.e., 1:3 serum dilution with 1:2 virus dilution) were in the second rows. The remaining serum dilutions in the wells were as follows: third rows with 1:18, fourth rows with 1:54, fifth rows with 1:162, sixth rows with 1:486, seventh rows with 1:1458, and eighth rows with 1:4374). Subsequently, 0.1 mL of the mixture of each well was transferred to the flat-bottom wells of Fc9 cell monolayer with 95-97% confluency and incubated for 24 h at 37° C. and 5% CO₂. The spent culture fluids were discarded. We then added 0.1 mL of 0.25% sterile methyl cellulose in EMEM with 5% heat-inactivated FBS to each well of the plate, and incubated at 37° C. in 5% CO₂ for 18 h or until the 35-50 virus plaques per well were observed in the duplicate virus control wells. The FCoV2 plaques in the wells were inactivated and stained with 0.1 mL/well of 1% crystal violet in 100% methanol at RT for 10 min. We then added 0.15 mL of 1% crystal violet in 20% methanol in each well, incubated at RT for 24 h, decanted, and removed the stain with water. The RBD blocking assay against FCoV2 is a modification of the above FCoV2 NAb assay. This assay differs from NAb assay by the following three features: 1) A set amount of RBD is used instead of cat serum. 2) FCoV2 dose was 4 TCID₅₀ instead of 2 TCID₅₀ to assure 100% cytopathic effect (CPE) on the Fc9 cells. 3) The flat-bottom plates with Fc9 cells at 98-100% confluency instead of 95-97% confluency were used at the time of adding the virus mixture.

Molecular Weights of RBDs: Based on the gel analysis using Coomassie Blue staining and anti-penta His monoclonal antibody (MAb)-treated RBD-immunoblot (data not shown), the approximate molecular weights of the coronavirus RBDs have been determined and confirmed also by the banding patterns of the immunoblots at 10% Tris-HCL gel with 30% acrylamide/bis compared to 40% acrylamide/bis. Both the gel staining and immunoblot results contained the RBDs with histidine (His)-tag sequence. These results demonstrate that the SCoV2 RBDs with tag (SEQ ID NO: 2), FCoV1 (SEQ ID NO: 6), and FCoV2 (SEQ ID NO: 7) are all highly glycosylated (Fig G1B, glycan MW is subtraction of two MW). All of these RBD pDNA, except for MB-RBD plasmid, expressed 70 aa tag residues (7,454 kDa) resulting from the HRV cleavage site, 8× His-Tag, and streptavidin-binding peptide (SBP)-Tag. Hence, approximately 7,454 kDa should be subtracted from all RBDs that used the same plasmid for expression. The approximate molecular weight (MW) of the RBD alone (minus tag sequences) are the following for SCoV2 UF (gp30; SEQ ID NO: 1), FCoV1 (gp52; SEQ ID NO: 5), and FCoV2 (gp59; SEQ ID NO: 7).

For example, SCoV2 Wuhan UF-RBD without 70 aa tag residues will result in 211 aa residues of 23,750 kDa without glycosylation, and with glycosylation about 29,564 kDa (round up to gp30) (SEQ ID NO: 1 with tag and 5 without tag). The SCoV2 Wuhan MB-RBD without two tags of 16 aa residues and 6× His) will result in a peptide of 223 aa residues which has a MW of 25,099 kDa without glycosylation and with glycosylation of about 30,992 kDa (gp31; SEQ ID NO: 3). Although the number of aa residues is higher with SCoV2 MB-RBD, the UF-RBD has higher MW on the gel and immunoblot (data not shown) due to the 70 aa sequence of the tags. The FCoV1 RBD has a solid band separately above the heavily glycosylated thick band as shown with a red arrow (data not shown).

### Example 1—Crossreactivity of FCoV2 Antisera with SCoV2 RBD Peptides

The following example describes serological assays performed to detect FCoV2 seroconversion in FCoV naturally transmitted laboratory cats and cross reactivity of FCoV2 antisera with SCoV2 RBD (SEQ ID NO: 1, 3, 9 and 10).

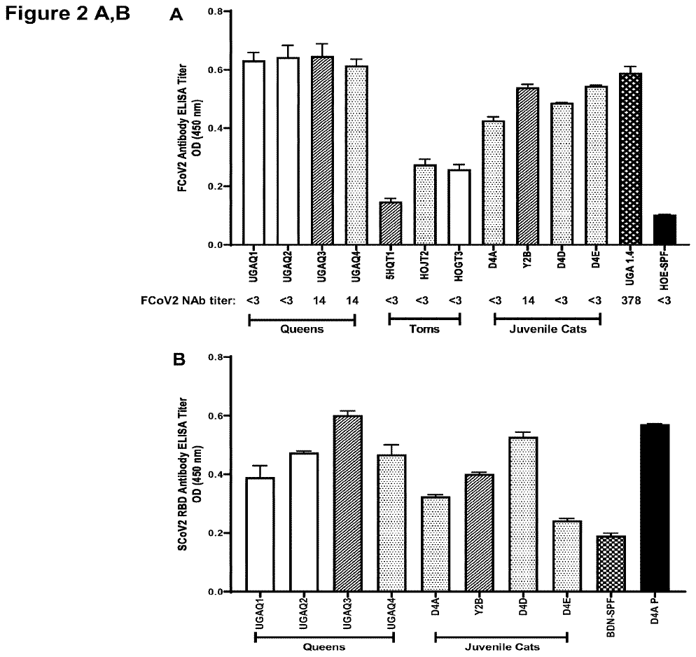

FCoV2 whole-virus and SCoV2 RBD ELISA: Studies on FCoV were initiated when three SPF inbred toms at a UF laboratory developed minor episode(s) of diarrhea after accidentally mating with four laboratory queens from UGA. The four juvenile laboratory cats born from two UGA queens (UGAQ3, UGAQ4) had FCoV antibodies at 12 and 16 weeks of age based on FCoV2 whole-virus ELISA. The UGA queens were all seropositive for FCoV2 (FIG. 2A) and the sera from the toms, after mating, were weakly seropositive for FCoV2 (FIG. 2B). Anti-sera from three of the four juvenile cats cross-reacted moderately with the SCoV2 RBD tested by SCoV2 RBD ELISA (FIG. 2B).

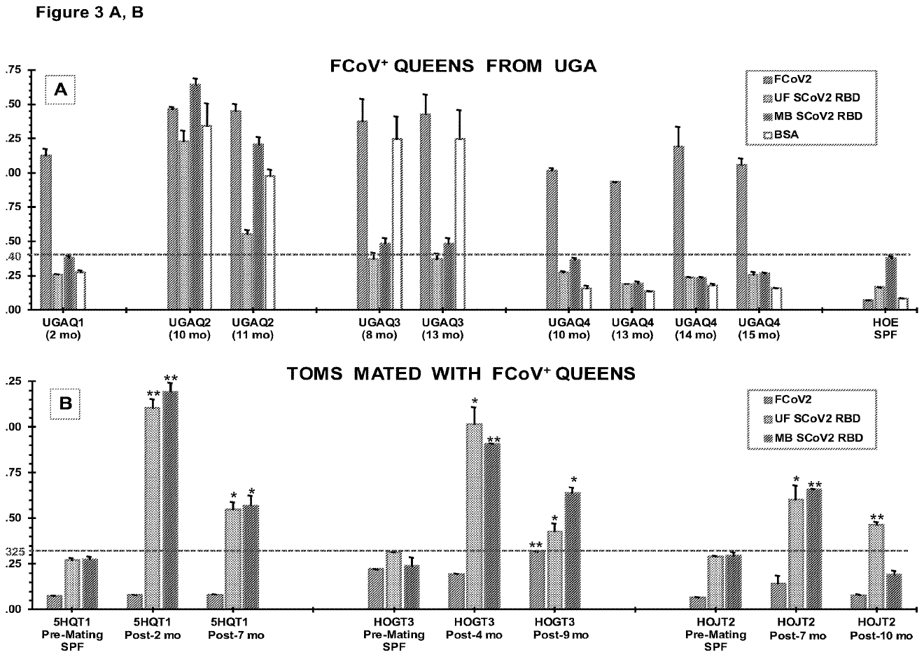

Stringent FCoV whole-virus ELISA and SCoV2 RBD ELISA: Two UGA queens (UGAQ1, UGAQ4) had high levels of serum reactivity with FCoV but no reactivity with BSA and SCoV2 RBDs (SEQ ID NOs: 1, 2, and 5), while the other two queens (UGAQ2, UGAQ3) had high serum reactivity with FCoV, which was slightly higher than the serum reactivity with BSA (FIG. 3A). The sera from UGAQ2 also had substantial cross-reactivity with both SCoV2 RBDs, whereas the sera from UGAQ3 had modest cross-reactivity with MB-RBD and below the threshold cross-reactivity with UF-RBD.

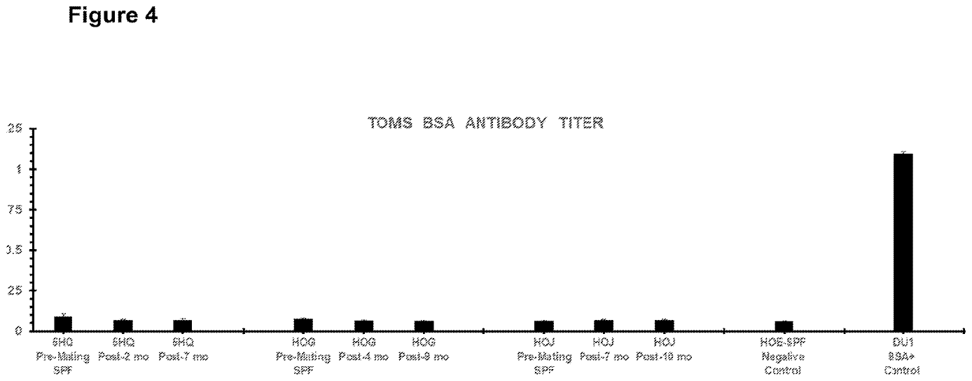

As expected, the sera from all toms before mating had no reactivity with FCoV, both UF/MB-RBDs (FIG. 3B), and BSA (BSA, Figure. 4). However, post-mating sera from all toms had significant reactivity with SCoV2 RBDs but not with FCoV, when compared to the corresponding pre-mating control results. The highest titers to SCoV2 RBDs were observed at the earliest time point of serum collection, closest to the first exposure to the FCoV-positive queen. Subsequent sera showed declines which were still significantly different from the pre-mating-serum titers (p<0.05). The decline suggested that the development of cross-reactive antibodies to SCoV2 RBD could have occurred during active FCoV infection. A slight conflict was observed between the substantial reactivity with FCoV for the toms (FIG. 2A) and no reactivity with FCoV for the toms (FIG. 3B). Overall, the sera from all three toms cross-reacted strongly with SCoV2 RBD by a stringent ELISA. Only one long-term FCoV infected queen UGAQ2 also had high titer of cross-reactive antibodies to SCoV2 RBD (FIG. 3A).

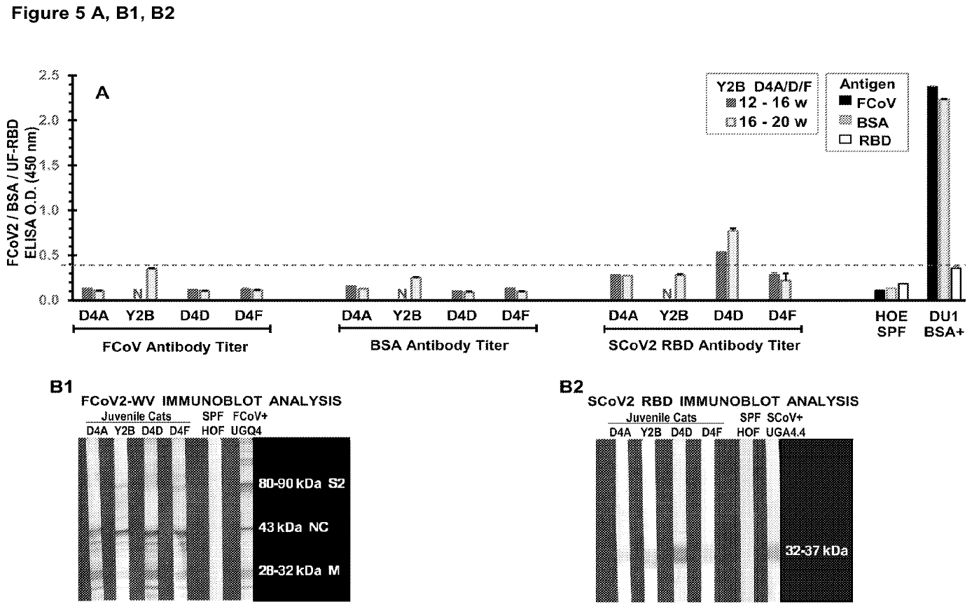

Juvenile cat studies: The juvenile cats were most likely seropositive for FCoV either by passive maternal FCoV antibodies from their FCoV-infected mothers (UGAQ3, UGAQ4) or became actively infected with FCoV transmitted from their mother. It has been reported that kittens weaned away from their FCoV-infected mothers at 4-5 weeks of age remain free of FCoV infection, but not those after 5 weeks of age.²⁴. The sera from one juvenile cat (Y2B), born from UGAQ3, and three juvenile cats (D4A, D4D, D4F), born from UGAQ4 (Table 1), were monitored for FCoV and SCoV2 RBD antibodies at 12-16 weeks and 16-20 weeks, respectively. Only D4D showed a sign of active FCoV infection based on the increase in antibodies cross-reacting with SCoV2 RBD by ELISA (FIG. 5A). Immunoblot analysis results for serum reactivity to SCoV2 RBD from D4D demonstrates stronger cross-reactivity to SCoV2 RBD, when compared to those of the remaining three juvenile cats (FIG. 5B).

### Example 2—Identification of FCoV1 and FCoV2 RBD Peptides

The following example describes the identification, selection and production of the FCoV1 and FCoV2 RBD peptides that were used in the serological assays.

FCoV1 and FCoV2 RBDs: The location for FCoV1 and FCoV2 RBDs has recently been predicted to be around residues 526-676, based on RBD sequence locations of porcine enteric diarrhea virus (PEDV) RBD at B residues 510-640 and testicular gastrointestinal enteric virus (TGEV) RBD at D3 residues 500-651.²⁵⁻²⁷ The TGEV RBD has been reported to bind to the porcine APN as its primary host cell receptor,^{27, 28} but whether PEDV RBD binds to pAPN as its host cell receptor is still controversial.^{26,29} The RBD sequence prediction of both PEDV and TGEV was based on monoclonal antibody (MAb) studies, identifying the most potent neutralizing MAb(s) to these porcine alphacoronaviruses reacting to B and D3 residue regions, respectively.^{26,27}

Since species-specific ACE2 is used by SCoV2 to infect cats, dogs, and humans, we reasoned that the FCoV1 and FCoV2 RBDs could be identified by aligning the UF-RBD and MB-RBD sequences against four known FCoV1 S1 sequences and against four known FCoV2 S1 sequences. The Wuhan SCoV2 RBD sequence is distinctly different in aa sequence from the RBD sequences of FCoV1 (11.5% & 12.3% identity; 31.8% & 33.6% similarity) and FCoV2 (12.2% & 12.3% identity; 36.5% & 37.7% similarity), the first value is based on UF-RBD, and the second value is based on MB-RBD. Hence, the locations for our proposed FCoV2 and FCoV1 RBDs may be in the vicinity exposed to the effect of immune pressure.

The full length aa sequence comparison of S glycoproteins of the SCoV2 Wuhan strain and FCoV2 WSU79-1146 strain displays the S1/S2 cleavage site for SCoV2 at a location different from the S1/S2 cleavage site for FCoV2.30-32 In addition, the SCoV2 RBD sequence alignment pattern with single FCoV2 RBD sequence of the full length S protein sequence differs slightly from the one aligned with four FCoV2 RBD sequences (data not shown), even though both analyses used the JusBio alignment server. The full-length S sequences between SCoV2 and FCoV2 show the RBD has the least sequence similarity followed by the NTD and then C-terminal domain with the most similarity or conservation among two S1 domains (Table 3). This pattern was the same whether the S1/S2 cleavage site for SCoV2 or FCoV2 was used. Furthermore, S2 sequence had the most aa sequence conservation between SCoV2 Wuhan and FCoV2 WSU79-1146.

TABLE 3
Summary of amino acid (aa) sequence identity and similarity between SCoV2 and FCoV2.

| | | | RBD | CTD | CTD | S1 | S1 | S2 | S2 |
|---|---|---|---|---|---|---|---|---|---|
| | NTD | RBD | (Proposed) | (SCoV2) | (FCoV2) | (SCoV2) | (FCoV2) | (SCoV2) | (FCoV2) |
| Total No. of | 370 | 290 | 273 | 147 | 297 | 834 | 984 | 654 | 504 |
| AA | | | | | | | | | |
| No. of AA | 55 | 38 | 36 | 24 | 64 | 121 | 161 | 210 | 170 |
| with Identity | | | | | | | | | |
| No. of AA | 155 | 107 | 100 | 69 | 151 | 345 | 427 | 409 | 326 |
| with Similarity | | | | | | | | | |
| % Identity | 14.9% | 13.1% | 13.2% | 16.8% | 21.5% | 14.5% | 16.4% | 32.1% | 33.7% |
| % Similarity | 41.9% | 36.9% | 36.6% | 54.7% | 50.8% | 41.4% | 43.4% | 62.5% | 64.7% |

Next, the full-length S sequences of FCoV1 UCD-1 strain were compared to the S sequence of the SCoV2 Wuhan strain (Table 4) to determine if similar changes occur as those observed when comparing the SCoV2 and FCoV2 (Table 3). Major changes in gap location are observed on the SCoV2 sequence when compared to the single FCoV1 sequence or to the four FCoV1 sequences (data not shown). The S1/S2 cleavage site for SCoV2 is only 46 aa plus five gaps away from the counterpart S1/S2 cleavage site for FCoV1.^{30, 31} Conversely, the FCoV1 S1/S2 cleavage site is 37 aa plus 14 gaps from the counterpart SCoV2 cleavage site.³⁰ Thus, the S1/S2 cleavage sites are in closer proximity between SCoV2 and FCoV1 than those between SCoV2 and FCoV2. The most aa sequence conservation is observed at the S2 sequence, followed by S1 CTD, S1 NTD, and then S1 RBD, which is identical in pattern to those observed between SCoV2 and FCoV2. These results suggest that NTD between SCoV2 and FCoV1/FCoV2, with the second least similarity (Table 4), may be structurally also exposed in the trimeric configuration allowing for more changes due to host immune pressure.^{33, 34}

TABLE 4
Summary of amino acid (aa) sequence identity and similarity between SCoV2 and FCoV1.

| | | | RBD | CTD | CTD | S1 | S1 | S2 | S2 |
|---|---|---|---|---|---|---|---|---|---|
| | NTD | RBD | (Proposed) | (SCoV2) | (FCoV1) | (SCoV2) | (FCoV1) | (SCoV2) | (FCoV1) |
| Total No. of | 402 | 307 | 279 | 149 | 97 | 885 | 833 | 639 | 691 |
| AA | | | | | | | | | |
| No. of AA | 46 | 39 | 33 | 23 | 17 | 108 | 114 | 203 | 209 |
| with Identity | | | | | | | | | |
| No. of AA | 154 | 111 | 101 | 64 | 50 | 330 | 344 | 417 | 431 |
| with Similarity | | | | | | | | | |
| % Identity | 11.4% | 12.7% | 11.8% | 15.4% | 17.5% | 12.2% | 13.7% | 31.8% | 30.2% |
| % Similarity | 38.3% | 36.2% | 36.2% | 42.9% | 51.5% | 37.3% | 41.3% | 65.3% | 62.4% |

Overall, the above results from sequence analyses suggest that the RBD for SCoV2 infection of cats may reside at a similar site as the RBD for SCoV2 infection of humans, with possibly similar contact aa residues in the receptor binding motif (RBM) to react to fACE2 and hACE2, respectively.

### Example 3—Detection of Cross Reactivity of Cat Anti-Sera with FCoV2 RBD by Immunoblotting

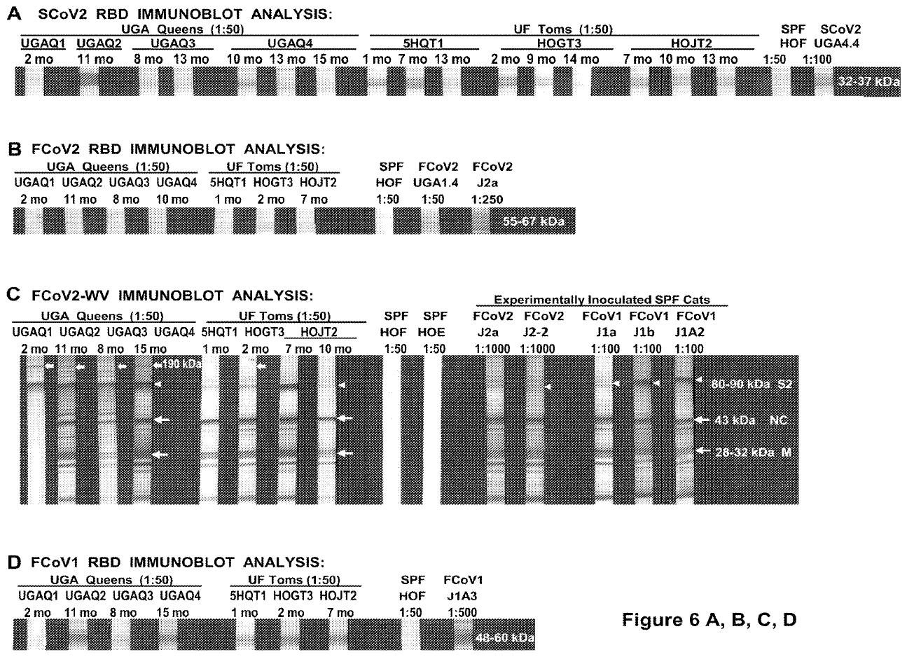

FCoV2 RBD (SEQ ID NO. 7), with the most cross-reactive sectional regions overlapping with SCoV2 RBD (SEQ ID NO: 1, SEQ ID NO: 3, SEQ ID NO: 9 and SEQ ID NO: 10) was produced in the same expression (EXPI293F cells) system as SCoV-2 UF-RBD (SEQ ID NO: 1), purified similarly as SCoV2 UF-RBD (SEQ ID NO: 1) in PBS, and used to develop the immunoblot strips. Two sera (UGAQ2, UGAQ4) from the four queens and the serum from all three toms strongly cross-reacted with the SCoV2 UF-RBD (FIG. 6a) but none of them reacted with FCoV2 RBD (FIG. 6B). All sera from the queens reacted with FCoV2 whole-virus immunoblot strips strongly at the membrane (M, 28-32 kDa), nucleocapsid (NC, 43 kDa), degraded spike S2 (80-90 kDa), and whole spike S (190 kDa) (FIG. 6C), with weaker bands at 20 kDa, 22 kDa, 40 kDa, and 55 kDa except of UGAQ1. The single serum available from UGAQ1 was collected when she was severely sick from uteritis and on antibiotics. Her serum only reacted to FCoV2 whole-virus at 90kd and higher (FIG. 6C) but not to SCoV2 (FIG. 6A), FCoV2 (FIG. 6B), and FCoV1 (FIG. 6D) RBDs. The toms had reactivity to NC, M, and proteins at 10 kDa, 20 kDa and 22 kDa but no reactivity to whole-virus S proteins and bands above 90 kDa except for HOGT3 at 2 months post-mating. The same sera from the toms did not react to FCoV2 RBD except for one tom (HOGT3) which reacted weakly with FCoV2 RBD (FIG. 6B) and without FCoV2 NAb titer (FIG. 2A) but strongly with FCoV1 RBD (FIG. 6D). Preliminary results demonstrate that the serum from three UGA queens (UGAQ2, UGAQ3, UGAQ4) and all three toms reacted with FCoV1 RBD (FIG. 6D). The nil-to-weak cross-reactivity to FCoV2 RBD and strong reactivity to FCoV1 RBD suggest that our toms and queens were infected with FCoV1 which is the most common serotype in the U.S.

The observation that FCoV1 infected cats cross-reacting with SCoV2 RBD was unexpected, since the amino acid sequence similarity is slightly more conserved between SCoV2 and FCoV2 than with FCoV1 (data not shown). Their sera also had nil-to-minimum titers of FCoV2 neutralizing antibodies which further supports FCoV1 infection of our queens and toms (FIG. 2A).

The sera from all four queens and three toms were evaluated for their cross-reactivity to SCoV2 UF-RBD immunoblot strips. Only sera from UGAQ2 and all three toms had strong cross-reactivity to SCoV2 RBD. Note that UGAQ4 was moderately cross-reactive to SCoV2 RBD only at the first blood collection (10 mo post-mating). The fact that the sera from UGAQ2 and all three toms strongly reacted with SCoV2 UF-RBD further confirms that their sera do cross-react with SCoV2 UF-RBD (SEQ ID NO: 1) and MB-RBD (SEQ ID NO: 3) even under stringent ELISA (FIG. 3A: UGAQ2; FIG. 2B: 5HQT1, HOJT2, HOGT3). The cross-reactive antibodies reacted to SCoV2 RBD immunoblot strips produced under a reducing condition, which suggests that these antibodies are reacting to linear peptide(s), glycan, or both.

### Example 4—In Vitro FCoV2 Infection Blocking Activity by FCoV2 RBD and SCoV2 RBDs

The following Example determines whether the FCoV2 RBD is capable of blocking the FCoV2 infection of the feline cell line (Fc9 cells). Duplicate wells, starting at 2.86 μg/mL, were serially diluted three-fold for each well, up to the titer of 0.106 μg/mL (fourth duplicate wells) and presented <50% cytopathic effect (CPE), whereas all wells for virus control had 100% CPE including the PBS controls. Thus, one TCID₅₀ titer, the titer with 50% CPE, is between 0.035-0.106 μg/mL (data not shown). Another assay demonstrated 1 TCID₅₀ titer of 50% blocking observed at about 0.100 μg/mL of FCoV2 RBD (data not shown). The SCoV2 RBD at 64.2 μg/mL blocked 100% of FCoV2 infection (first duplicate wells), whereas 42.8 μg/mL blocked 30% CPE. Both FCoV2 and SCoV2 RBDs caused no cellular toxicity (data not shown). All plates were photographed at 100% brightness and 65% contrast. Overall, 0.1-1.0 μg/mL of FCoV2 RBD blocked 50%-100% of FCoV2 infection, respectively; whereas 48-64 μg/mL of SCoV2 RBD cross-blocked 50%-100% of FCoV2 infection, respectively, at doses without any cellular toxicity. The data provided in this Example confirms that FCoV2 RBD sequence (SEQ ID NO: 8) includes the RBD site associated with FCoV2 infection.

### DISCUSSION

Cross-reactive antibodies to SCoV2 RBD developing in cats during active FCoV infection was totally unexpected. The peak sera from UGAQ2 and all three toms cross-reacted strongly with sensitive SCoV2 RBD ELISA and immunoblot, but not with FCoV2 RBD (FIGS. 6A and 6B). This observation indicated that the cats were most likely infected with FCoV1. In addition, all toms lacked FCoV2 whole-virus ELISA titer except for one tom (HOGT3) at post-9 months FCoV contact infection (FIG. 3). This observation supported the almost nil-to-low cross-reactivity with FCoV2 whole-virus antigen in the less stringent FCoV2 ELISA, using overnight serum incubation at 1:100 dilution (FIG. 2A). At 1:50 serum dilution of FCoV2 whole-virus immunoblot analysis, the sera from the toms also reacted to FCoV2, but only minimally-to-moderately at NC and M but not at S glycoproteins. This suggests that FCoV infection was most likely low in titer in these toms. In addition, it has been reported that M glycoproteins are expressed most during in vitro infection followed by NC proteins and then S glycoproteins^{35,36}. Hence, the FCoV2 antigen levels on the immunoblot may also reflect such antigen distribution.

Perhaps the most striking observation was the sera from the toms cross-reacting to SCoV2 RBD (SEQ ID NO: 1) without reacting to FCoV2 S glycoprotein on the FCoV2 whole-virus immunoblot (FIGS. 6A and 6C). These results indicate that cross-reactive antibodies to SCoV2 RBD appeared without major development of cross-reactive antibodies to FCoV2 S glycoprotein, including S2 glycoprotein at most timepoints, if they were indeed infected with more common FCoV1. None of the cats (queens, toms, and their first-generation kittens) developed major neutralizing antibodies to FCoV2 based on FCoV2 WSU79-1146 neutralization assay on feline Fc9 cell line (FIG. 6D). This result further supports that the cats were infected with FCoV1. The fact that the immunoblots used in the experiments described herein were developed under reducing conditions suggests that the cross-reacting epitopes on SCoV2 RBD are either linear amino acid epitopes and/or glycosylated epitope(s).

The lack of serum cross-reactivity with FCoV2 Spike glycoproteins by the toms was unexpected since all four queens, including UGAQ2, reacted with FCoV2 S glycoprotein (FIG. 6C). Since S2 glycoprotein has high amino acid sequence identity and similarity, the likelihood of the cross-reactivity with S2 of the S glycoprotein may be a strong possibility based on the study by Zhao et al. 2007.³³ Table 5 below provides a summary of similarity and sequence identity of SCoV1 and SCoV2 S1 and S2 spike glycoproteins.

TABLE 5
Summary of Amino Acid (AA) Sequence Identity and Similarity
between SCoV2 and SCoV1 S1 or S2 spike glycoproteins.

| | NTD | RBD | CTD | S1 | S2 | Spike |
|---|---|---|---|---|---|---|
| Total No. of | 322 | 211 | 144 | 676 | 588 | 1277 |
| AA | | | | | | |
| No. of AA | 164 | 154 | 111 | 438 | 529 | 967 |
| with Identity | | | | | | |
| No. of AA | 259 | 190 | 132 | 594 | 579 | 1173 |
| with Similarity | | | | | | |
| % Identity | 50.9% | 73.0% | 77.1% | 64.8% | 90.0% | 75.7% |
| % Similarity | 80.4% | 90.0% | 91.7% | 87.9% | 98.5% | 91.9% |

The analysis herein shows the NTD as the least conserved domain with 80.4% similarity, and the S2 glycoprotein as the most conserved domain with 90% identity and 98.5% similarity. Our comparative analysis also shows RBD to be slightly more conserved than NTD but still less conserved than the CTD. Therefore, our finding that FCoV-infected cats develop cross-reacting antibodies to SCoV2 RBD was greatly unexpected when their study showed no cross-reactivity to SCoV1 S1 glycoprotein, since SCoV1 and SCoV2 have 64.8% identity and 87.9% similarity (Table 5).

Remarkably, the study demonstrated that SCoV2 UF-RBD at a high concentration of 48 μg/mL was able to cross-block or cross-protect against FCoV2 infection of feline cell line at 1 TCID₅₀ dose without any cellular toxicity. The ability of FCoV2 and SCoV2 RBDs to block in vitro FCoV2 infection also suggest that these RBDs may be important for developing an effective pan-coronavirus vaccine for pet animals such as cats, dogs, and hamsters. SCoV2 infected hamsters from Europe imported to pet shops in Hong Kong have been reported as a source of two separate hamster-to-human transmissions and subsequent human-to-human transmission of SCoV2 Delta variant, with the sequence found predominantly in Europe.³⁷ Inoculation of laboratory Syrian golden hamsters resulted in infection of the hamsters with major loss in weight, lung infection, and respiratory disease.³⁸⁻⁴²

Profound acute SCoV2 infection of humans causes gastrointestinal (GI) manifestation (diarrhea, vomiting, nausea, abdominal pain), including longer fecal shedding than those detected in the nasopharyngeal samples.⁴³⁻⁴⁶ Both FCoV and CCoV also cause GI tract disease in their respective animal hosts and clinically affect the kittens and puppies more than adults, with the exception of FIPV disease.^{5,44,47} FCoV2 RBD sequence alignment comparison of CCoV serotype 2 (CCoV2) and FCoV2 RBDs shows 95.9% amino acid sequence similarity and 87.7% amino acid sequence identity (Table 6).

TABLE 6
Summary of Amino Acid (AA) Sequence Identity and Similarity between CCoV2 and FCoV2.

| | | | CTD | CTD | S1 | S1 | S2 | S2 |
|---|---|---|---|---|---|---|---|---|
| | NTD | RBD | (KRK) | (KRKYR) | (KRK) | (KRKYR) | (KRK) | (KRKYR) |
| Total No. of | 390 | 268 | 267 | 269 | 960 | 962 | 493 | 491 |
| AA | | | | | | | | |
| No. of AA with | 127 | 235 | 246 | 247 | 637 | 638 | 464 | 463 |
| Identity | | | | | | | | |
| No. of AA with | 229 | 257 | 264 | 265 | 782 | 783 | 485 | 484 |
| Similarity | | | | | | | | |
| % Identity | 32.6% | 87.7% | 92.1% | 91.8% | 66.3% | 66.3% | 94.1% | 94.3% |
| % Similarity | 58.7% | 95.9% | 98.9% | 98.5% | 81.6% | 81.4% | 98.4% | 98.6% |

The high sequence similarity between FCoV2 and CCoV2 RBDs may explain why CCoV2 can infect fAPN expressing feline cells²⁸. Although FCoV1 and CCoV1 RBDs possess only amino acid sequence identity of 55.5%, their sequence similarity of 80.8% is remarkably high (Table 7), suggesting that they have a common lineage with evolutionary changes and perhaps also by sectional recombination.^{34,43,48}

TABLE 7
Summary of Amino Acid (AA) Sequence Identity and Similarity between CCoV1 and FCoV1

| | | | CTD | CTD | S1 | S1 | S2 | S2 |
|---|---|---|---|---|---|---|---|---|
| | NTD | RBD | (CCoV1) | (FCoV1) | (CCoV1) | (FCoV1) | (CCoV1) | (FCoV1) |
| Total No. of | 435 | 281 | 88 | 86 | 800 | 798 | 675 | 677 |
| AA | | | | | | | | |
| No. of AA | 150 | 156 | 58 | 58 | 375 | 375 | 496 | 496 |
| with Identity | | | | | | | | |
| No. of AA | 253 | 227 | 71 | 71 | 567 | 567 | 603 | 603 |
| with Similarity | | | | | | | | |
| % Identity | 34.5% | 55.5% | 65.9% | 67.4% | 46.9% | 47.0% | 73.5% | 73.3% |
| % Similarity | 54.2% | 80.8% | 80.7% | 82.6% | 70.9% | 71.0% | 89.3% | 89.1% |

The most dissimilar sequence region is in the NTD of the S1 sequences between the FCoV1 and CCoV1 and between the FCoV2 and CCoV2, while the most conserved region is found beyond RBD at the carboxyl-end of S1 and with the S2 sequences (Tables 6 and 7). These findings suggest that a composition developed by combining FCoV1 RBD (SEQ ID NO: 5) and FCoV2 RBD (SEQ ID NO: 7) together with SCoV2 RBD (SEQ ID NO: 1, SEQ ID NO: 3 and SEQ ID NO: 10), optionally including the addition of CCoV1/CCoV2 (SEQ ID NOs: 11 and 12, respectively) RBDs, is effective in inhibiting SCoV2 infection in cats, dogs, and hamsters.

### Example 5—Anti-CoV RBD IFNγ and IL-2 ELISpot Responses

Anti-CoV RBD IFNγ and IL-2 ELISpot responses of the peripheral blood mononuclear cells (PBMC) from a COVID-19 vaccinated subject Y3. Subject Y3 has been vaccinated five-times with COVID-19 vaccine since 2020-2022. The last vaccination consisted of Moderna bivalent COVID-19 vaccine but those prior to this vaccine were all monovalent Pfizer COVID-19 vaccine. The IFNγ and IL-2 responses to human (SCoV2) and feline (FCoV1, FCoV2) receptor binding domains (RBDs) are shown with the months post last vaccination, when the blood was collected for PBMC isolation. Subject Y3 received five vaccination and therefore this subject's IFNγ responses to all RBDs appeared to be hyperreactive at 4 mo post 5th vaccination (p5v) and completely gone by 9 mo p5v. However, the IL-2 ELISpot responses suggest that vaccine-induced IL-2 responses still remain at 10 mo p5v. The cytokine IL-2 is known as a strong T-cell immune mediator and not known as an inflammatory cytokine. The red dotted line represents the threshold of the average PBS control response. Since IFNγ is well known to be inflammatory cytokine as well as a moderate dose to be important T cell immune modulator. Current results suggest that COVID19 vaccine-induced IFNγ for this subject is responding as inflammatory cytokine that crashes in response by 9 mo p5v. Those responses to RBD(s) substantially above this threshold would indicate a substantial or significant PBMC response. The statistical significant difference between the PBS control and the respective responses to the RBDs is shown as p<0.025 (*) and p<0.05 (**), based on two-tailed paired T-Test. This subject has never been infected with SCoV2. The description of this subject's profile is shown in the table below.

| Subject Code | Y2 | Y3 | Y7 | Y8 | Y9 | Y10 |
|---|---|---|---|---|---|---|
| Age in Years | 69 | 69 | 72 | 48 | 48 | 21 |
| Gender | F | F | F | M | M | M |
| Vaccination Freq | 4 | 5 | 5 | 3 | 3 | 4 |
| Vaccine Type** | M/M/M/Mb | P/P/P/P/Mb | P/P/P/P/Mb | M/P/Pb | P/P*/P | P/P/P/P |
| SCoV2 Exposure | 0 | 0 | 0 | 0 | 1* | 0 |
| FCoV Cat | + | − | − | − | − | + |
| Exposure | | | | | | |

*Subject Y9 was infected with SCoV2 at 1 year post the second Pfizer COVID19 vaccination.
**Both Pfizer monovalent and bivalent vaccines are used at a total amount of 30 μg/dose. Both Moderna monovalent and bivalent vaccines are used at a total amount of 50 μg/dose.
Abbreviations: Moderna monvalent (M), Moderna bivalent (Mb), Pfizer monovalent (P), Pfizer bivalent (Pb)

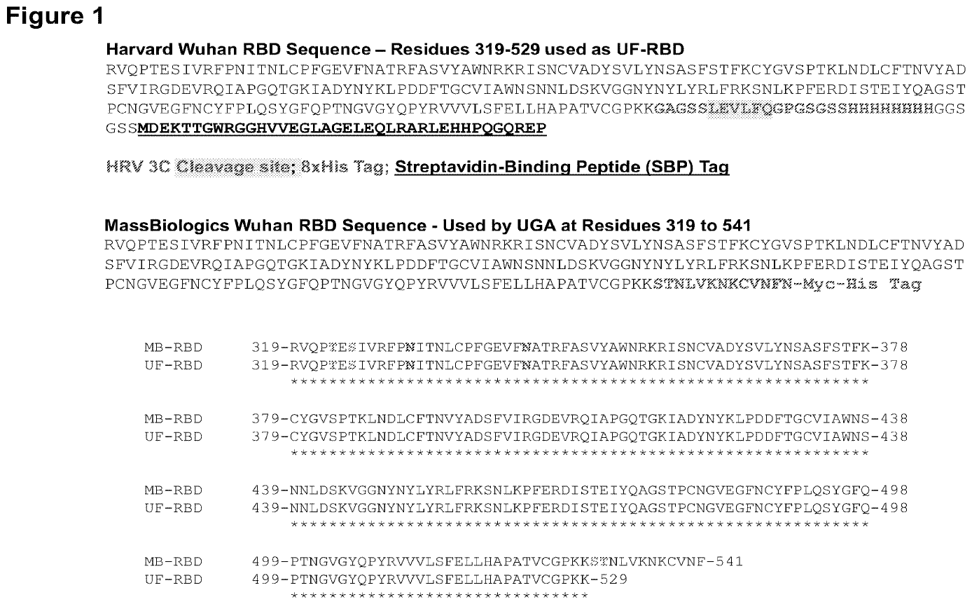

Three COVID-19 vaccinated subjects (Y2, Y8, Y10) were reported to be free of SCoV2 exposure, whereas one subject Y9 was infected with SCoV2 one year after the second vaccination which is also about three month before the third vaccination. The blood was collected from Y9 at 12 months post last 3rd vaccination or 1.5 years post SCoV2 infection. Y2 and Y10 have multiple FCoV1 infected cats which may have altered their COVID-19 vaccination results by constant shedding of FCoV1 in their household. In contrast, vaccinated/infected Y9 has no FCoV1 infected cats at household but has significant IFNγ responses to FCoV1 and FCoV2 RBD but not to SCoV2 RBD when his last vaccination was 3.5 mo since blood collection for the PBMC used in the assay. Subject Y8 has no cats and has not been infected with SCoV2. This subject who is same age as Y9 (48 years-old) had no responses to any RBDs even though he was last vaccinated 9 mo ago with Pfizer bivalent. In contrast subject Y10, who was vaccinated with Pfizer monovalent 9 mo before the blood collection for PBMC shown, had significant IFNγ responses to SCoV2 and FCoV1 RBDs and substantial IFNγ response to FCoV2 RBD. Y10, at the time of bleeding was 21 years-old, has retained the CoV RBD responses far better than Y8, Y9, and Y2, and even Y3 at 9 mo p5v (FIG. 1). Surprisingly, IFNγ responses to SCoV2 RBD are slightly-to-substantially lower levels than those to FCoV1 RBD for Y2, Y10, and Y9, and even Y3 at 10 mo p5v with IL-2 response. Such results suggest immunogen-specific downregulation of immune responses.

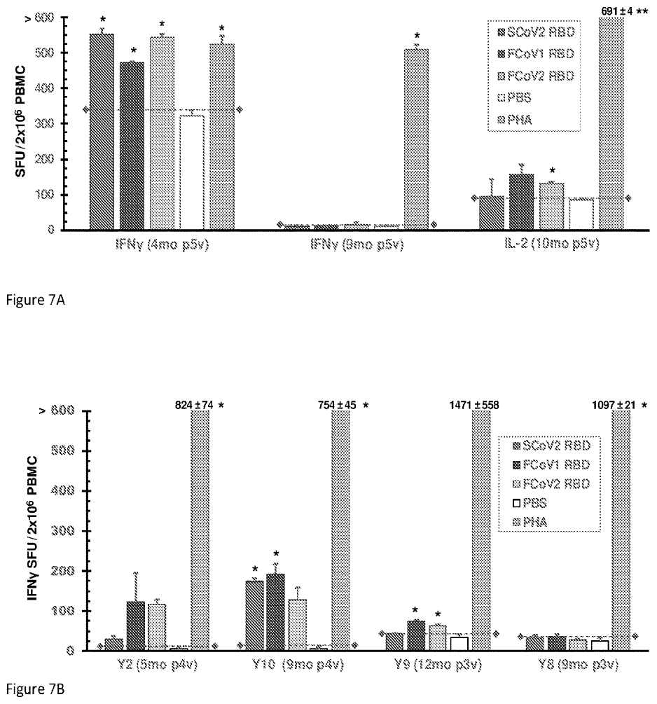

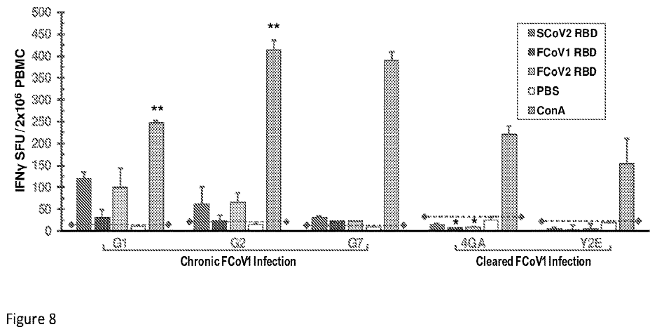

As shown in FIGS. 7A and 7B, COVID-19 vaccines induce FCoV1 and FCoV2 RBD responses more strongly than those to SCoV2 RBD. The dotted line for each subject represents the threshold of the average PBS control response. Reponses to RBD(s) that are substantially above this threshold indicates a substantial or significant PBMC response. The statistically significant difference between the PBS control and the respective responses to the RBD(s) are shown as p<0.05 (*), based on two-tailed paired T-Test.

FIG. 8 shows that chronically FCoV1-infected cats (G1, G2, G7) have antibodies to FCoV whole-virus and live together, causing constant reinfection. FCoV1-infection-cleared cats (4GA, Y2E) do not have antibodies to FCoV by living individually in single housing unit to prevent reinfection that occurs when housed together with one or more FCoV1-infected cats. The dotted line for each cat represents the threshold of the average PBS control response. These results suggest that downregulation of IFNγ responses to FCoV1 RBD in the infecting coronavirus FCoV1 occurs with cats G1, G2, and 4GA. This observation may be similar to the downregulation of IFNγ responses to SCoV2 RBD of Y2, Y10 and especially with subject Y9. Those responses to RBD(s) substantially above this threshold would indicate a substantial or significant PBMC response. The statistically significant difference between the PBS control and the respective responses to the RBD(s) are shown as p<0.05 (*) and p≤0.025 (**), based on two-tailed paired T-Test.

### REFERENCES

1. Holmes et al, Philos. Trans. R. Soc. Lond. B. Biol. Sci. 2004; 359 (1447): 1059-1065.
2. Herrewegh, et al. 1998. J. Virol. 72 (5), 4508-4514.
3. Horzinek M C, Osterhaus A D. The virology and pathogenesis of feline infectious peritonitis. Brief review. Arch Virol. 1979; 59 (1-2): 1-15.
4. Vennema H et al. 1998. Virology 1998; 243 (1): 150-157.
5. Pedersen, N. C. Vet J 2014; 201:123-132.
6. Krentz et al. Viruses. 2021; 13 (11): 2228. doi:10.3390/v13112228
7. Weiss et al. Comp Immunol Microbiol Infect Dis. 1981; 4 (2): 175-189.
8. Pedersen et al. (1984) American journal of veterinary research, 45 (12), 2580-2585.
9. Hohdatsu et al. Arch Virol. 1991; 120 (3-4): 207-217.
10. Zhu et al. Respir Res. 2020; 21 (1): 224.
11. Li et al., Sci Adv. 2020; 6 (27): eabb9153. Published 2020 Jul. 1.
12. Wardeh et al. Nat Commun. 2021; 12 (1): 780. Published 2021 Feb. 16.
13. Morens et al. N Engl J Med. 2022; 386 (4): 297-299.
14. McAloose et al. mBio. 2020; 11 (5): e02220-20. Published 2020 Oct. 13.
15. Newman et al. New York, March-April 2020. MMWR Morb Mortal Wkly Rep. 2020; 69 (23): 710-713. Published 2020 Jun. 12.
16. Sit et al. Nature. 2020; 586 (7831): 776-778.
17. Decaro et al. Front Vet Sci. 2021; 8:619207. Published 2021 Feb. 10.
18. OIE. (2021b). SARS-CoV-2 in animals. Situation report 1. OIE.
19. Sailleau et al. Transbound Emerg Dis. 2020; 67 (6): 2324-2328.
20. Segalés et al. Proc Natl Acad Sci USA. 2020; 117 (40): 24790-24793.
21. Nanishi et al. Sci Transl Med. 2022; January 26; 14 (629): eabj5305.
22. Yates J L, et al., Cell Rep Med. 2021, e1-4.
23. Kida K, et al., J Vet Med Sci. 1999; 61: 935-938.
24. Aillo S E, Moses M A. Feline Infectious Peritonitis: Management of Multicat Households, In: The Merck Veterinary Manual; Chapter: Generalized Conditions, 11th Edition, 2016. p 788.
25. Regan et al. Virology 2012; 430:90-99.
26. Li et al. J Virol 2017; 91 (12) e00273-17.
27. Reguera et al. J Gen Virol 2011; 92:1117-1126
28. Tusell et al. J Virology, 2007; 81:1261-1273
29. Shirato et al. J Gen Virol 2016; 97:2528-2539.
30. Coutard et al. Antiviral Res 2020; April; 176:104742.
31. Millet et al. Virus Res 2015; April 16; 202:120-34.
32. Follis et al., Virology. 2006 Jul. 5; 350 (2): 358-69.
33. Zhao et al. Viruses, 2019, 11, 743.
34. Jaimes et al. Viruses 2020, 12, 83.
35. Arya et al., J Mol Biol 2021; January 22; 233 (2) 166725.
36. Yan et al., Signal Transduct Target Ther 2022; January 27; 7 (1): 26.
37. World Organization for Animal Health: Global cases of SARS-CoV-2 in animals./www.oie.int/en/what-we-offer/emergency-and-resilience/covid-19/#ui-id-3 (accessed Mar. 27, 2022) (Report ID FUR_154029).
38. Sia et al., Nature. 2020 July; 583 (7818): 834-838.
39. Abdelnabi et al., EBioMedicine. 2021 June; 68:103403.
40. Osterrieder et al., Viruses 2020; July 20; 12 (7): 779.
41. Mohandas et al., Viruses 2021; September 5; 13 (9): 1773.
42. Rogers et al., Science. 2020; 369 (6506): 956-963.
43. Vlasova et al., Emerg Microbes Infect 2022; December; 11 (1): 699-702.
44. Licitra et al., Viruses 2014; August 22; 6 (8): 3363-76.
45. Hussain et al., Front Immunol. 2021 Oct. 14; 12:765965.
46. Díaz et al., Gastroenterol Hepatol. 2022 Jan. 22: S0210-5705 (22) 00001-2.
47. Stout et al, Veterinary Microbiology 2020; 247:108777.
48. Lednicky et al., Clin Infect Dis 2021; October 28: ciab924.

What is claimed is:

1. A composition comprising a nucleic acid encoding a feline coronavirus (FCoV) receptor binding domain (RBD) peptide and a severe acute respiratory syndrome coronavirus 2 (SCoV2) RBD peptide.

2. The composition of claim 1, wherein the FCoV RBD peptide is an FCoV serotype 2 (FCoV2) RBD peptide.

3. The composition of claim 2, wherein the FCoV2 RBD peptide comprises an amino acid sequence that is at least 70% identical to the amino acid sequence set forth in SEQ ID NO: 7.

4. The composition of claim 2, wherein the FCoV2 RBD peptide comprises the amino acid sequence set forth in SEQ ID NO: 7.

5. The composition of claim 1, wherein the FCoV RBD peptide is an FCoV serotype 1 (FCoV1) RBD peptide.

6. The composition of claim 5, wherein the FCoV1 RBD peptide comprises an amino acid sequence that is at least 70% identical to the amino acid sequence set forth in SEQ ID NO: 5.

7. The composition of claim 5, wherein the FCoV1 receptor binding domain peptide comprises an amino acid sequence set forth in SEQ ID NO: 5.

8. The composition of any one of claims 1-7, comprising a FCoV1 RBD peptide and a FCoV2 RBD peptide.

9. The composition of claim 1, wherein the SCoV2 RBD peptide comprises an amino acid sequence that is at least 70% identical to the amino acid sequence set forth in SEQ ID NO: 1, SEQ ID NO: 3, SEQ ID NO: 9, or SEQ ID NO: 10.

10. The composition of any one of claims 1-9, wherein the SCoV2 RBD peptide comprises an amino acid sequence set forth in SEQ ID NO: 1, SEQ ID NO: 3, SEQ ID NO: 9, or SEQ ID NO: 10.

11. The composition of any one of claims 1-10, comprising
(a) a FCoV1 RBD peptide comprising an amino acid sequence set forth in SEQ ID NO: 5 and a SCoV2 RBD peptide comprising an amino acid sequence set forth in SEQ ID NO: 1;
(b) a FCoV1 RBD peptide comprising an amino acid sequence set forth in SEQ ID NO: 5 and a SCoV2 RBD peptide comprising an amino acid sequence set forth in SEQ ID NO: 3;
(c) a FCoV1 RBD peptide comprising an amino acid sequence set forth in SEQ ID NO: 5 and a SCoV2 RBD peptide comprising an amino acid sequence set forth in SEQ ID NO: 9;
(d) a FCoV1 RBD peptide comprising an amino acid sequence set forth in SEQ ID NO: 5 and a SCoV2 RBD peptide comprising an amino acid sequence set forth in SEQ ID NO: 10;
(e) a FCoV2 RBD peptide comprising an amino acid sequence set forth in SEQ ID NO: 7 and a SCoV2 peptide comprising an amino acid sequence set forth in SEQ ID NO: 1;
(f) a FCoV2 RBD peptide comprising an amino acid sequence set forth in SEQ ID NO: 7 and a SCoV2 peptide comprising an amino acid sequence set forth in SEQ ID NO: 3;
(g) a FCoV2 RBD peptide comprising an amino acid sequence set forth in SEQ ID NO: 7 and a SCoV2 peptide comprising an amino acid sequence set forth in SEQ ID NO: 9;
(h) a FCoV2 RBD peptide comprising an amino acid sequence set forth in SEQ ID NO: 7 and a SCoV2 peptide comprising an amino acid sequence set forth in SEQ ID NO: 10;
(i) a FCoV1 RBD peptide comprising an amino acid sequence set forth in SEQ ID NO: 5, a FCoV2 RBD peptide comprising an amino acid sequence set forth in SEQ ID NO: 7, and a SCoV2 peptide comprising an amino acid sequence set forth in SEQ ID NO: 1;
(j) a FCoV1 RBD peptide comprising an amino acid sequence set forth in SEQ ID NO: 5, a FCoV2 RBD peptide comprising an amino acid sequence set forth in SEQ ID NO: 7, and a SCoV2 peptide comprising an amino acid sequence set forth in SEQ ID NO: 3;
(k) a FCoV1 RBD peptide comprising an amino acid sequence set forth in SEQ ID NO: 5, a FCoV2 RBD peptide comprising an amino acid sequence set forth in SEQ ID NO: 7, and a SCoV2 peptide comprising an amino acid sequence set forth in SEQ ID NO: 9; or
(l) a FCoV1 RBD peptide comprising an amino acid sequence set forth in SEQ ID NO: 5, a FCoV2 RBD peptide comprising an amino acid sequence set forth in SEQ ID NO: 7, and a SCoV2 peptide comprising an amino acid sequence set forth in SEQ ID NO: 10.

12. The composition of any one of claims 1-11, further comprising a nucleic acid encoding a canine coronavirus serotype 1 (CCoV1) receptor binding domain (RBD) peptide or nucleic acid encoding a canine coronavirus serotype 2 (CCov2) receptor binding domain (RBD) peptide.

13. The composition of claim 12, wherein the CCoV1 RBD peptide comprises the amino acid sequence set forth in SEQ ID NO: 11.

14. The composition of claim 12, wherein the CCoV2 RBD peptide comprises the amino acid sequence set forth in SEQ ID NO: 12.

15. The composition of any one of claims 1-14, comprising a nucleic acid encoding an amino acid sequence set forth in any one of SEQ ID NOs: 93-95.

16. The composition of any one of claims 1-15, wherein the composition further comprises an immunogen.

17. The composition of claim 16, wherein the immunogen comprises a nucleic acid encoding a conserved SCoV2 CD8+ T cell epitope peptide.

18. The composition of claim 17, wherein the conserved SCoV2 CD8+ T cell epitope peptide is an amino acid sequence set forth in any one of SEQ ID NOs: 13-15 and 19-43.

19. The composition of claim 16, wherein the immunogen comprises a nucleic acid encoding a conserved FCoV2 CD8+ T cell epitope peptide.

20. The composition of claim 19, wherein the conserved FCoV2 CD8+ T cell epitope peptide is an amino acid sequence set forth in any one of SEQ ID NOs: 16-18 and 44-67.

21. The composition of claim 19 or claim 20, wherein the conserved CD8+ T cell epitope peptide is a FCoV2 nucleocapsid protein.

22. The composition of claim 21, the FCoV2 nucleocapsid protein is an amino acid sequence set forth in any one of SEQ ID NOs: 16-18.

23. The composition of claim 17, wherein the conserved SCoV2 CD8+ T cell epitope peptide is a SCoV2 nucleocapsid protein.

24. The composition of claim 23, the SCoV2 nucleocapsid protein is an amino acid sequence set forth in any one of SEQ ID NOs: 13-15.

25. The composition of claim 17, wherein the conserved SCoV2 CD8+ T cell epitope peptide is a SCoV2 polymerase enzyme epitope peptide.

26. The composition of claim 25, wherein the SCoV2 polymerase enzyme epitope peptide is an amino acid sequence set forth in any one of SEQ ID NOs: 19-43.

27. The composition of claim 19, wherein the conserved CD8+ T cell epitope peptide is a FCoV2 polymerase enzyme epitope peptide.

28. The composition of claim 27, wherein the FCoV2 polymerase enzyme epitope peptide is an amino acid sequence set forth in any one of SEQ ID NOs: 44-67.

29. A composition comprising a nucleic acid encoding one or more SCoV2 M-protease epitope peptide.

30. The composition of claim 29, wherein the SCoV2 M-protease epitope peptide comprises an amino acid sequence set forth in any one of SEQ ID NOs: 68-75.

31. A composition comprising a nucleic acid encoding one or more FCoV1 M-protease epitope peptide.

32. The composition of claim 31, wherein the FCoV1 M-protease epitope peptide is an amino acid sequence set forth in any one of SEQ ID NOs: 76-83.

33. The composition of any one of claims 15-32, further comprising a nucleic acid encoding a T cell helper peptide.

34. The composition of claim 33, wherein the T cell helper peptide comprises an amino acid sequence set forth in any one of SEQ ID NOs: 85-88.

35. The composition of any one of claims 29-34, comprising a nucleic acid encoding an amino acid sequence set forth in any one of SEQ ID NOs: 89-92, and combinations thereof.

36. The composition of any one of claims 1-35, wherein the nucleic acid encoding the FCoV RBD peptide and the SCoV2 RBD peptide is in a vector.

37. The composition of any one of claims 16-36, wherein the nucleic acid encoding the T cell epitope peptide is in a vector.

38. The composition of any one of claims 1-37, wherein the composition is formulated in a nanoparticle, a liposome, a microparticle, a microsphere, a nanosphere, a unilamellar vesicle, a multilamellar vesicle, or a virus-like particle (VLP).

39. The composition of claim 38, wherein the composition is formulated in a lipid nanoparticle.

40. The composition of claim 39, wherein the lipid nanoparticle is T-cell specific.

41. The composition of claim 40, wherein the lipid nanoparticle is specific for a dendritic cell.

42. A vector comprising a nucleic acid encoding a feline coronavirus (FCoV) receptor binding domain (RBD) peptide and a severe acute respiratory syndrome coronavirus 2 (SCoV2) RBD peptide.

43. A vector comprising a nucleic acid encoding one or more SCoV2 M-protease epitope peptide.

44. The vector of claim 43, wherein the SCoV2 M-protease epitope peptide comprises an amino acid sequence set forth in any one of SEQ ID NOs: 68-75.

45. A vector comprising a nucleic acid encoding one or more FCoV1 M-protease epitope peptide.

46. The composition of claim 45, wherein the FCoV1 M-protease epitope peptide is an amino acid sequence set forth in any one of SEQ ID NOs: 76-83.

47. The vector of any one of claims 43-46, further comprising a nucleic acid encoding a T cell helper peptide.

48. The vector of claim 48, wherein the T cell helper peptide comprises an amino acid sequence set forth in any one of SEQ ID NOs: 85-88.

49. The vector of any one of claims 29-34, comprising a nucleic acid encoding an amino acid sequence set forth in any one of SEQ ID NOs: 89-92, and combinations thereof.

50. A method for treating or preventing a coronavirus infection in a subject in need thereof, the method comprising administering to the subject an effective amount of a first composition of any one of claims 1-15 and a second composition of any one of claims 16-41.

51. The method of claim 50, wherein the coronavirus infection is a SARS-CoV-2, FCoV1, FCoV2 or CCoV infection.

52. A method for inducing an immune response in a subject, the method comprising administering to a subject in need thereof an effective amount of the composition of a first composition of any one of claims 1-15 and a second composition of any one of claims 16-41.


SEQUENCE LISTING (98 sequences)

SEQ ID NO: 1 - protein, 211 aa, synthetic construct
RVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKK

SEQ ID NO: 2 - protein, 281 aa, synthetic construct
RVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKGAGSSLEVLFQGPGSGSSHHHHHHHHGGSGSSMDEKTTGWRGGHVVEGLAGELEQLRARLEHHPQGQREP

SEQ ID NO: 3 - protein, 223 aa, synthetic construct
RVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNF

SEQ ID NO: 4 - protein, 239 aa, synthetic construct
RVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFEQKLISEEDLHHHHHH

SEQ ID NO: 5 - protein, 279 aa, synthetic construct
FGKTMHSYFCFANFSNSMVRQFLGILPPTVREFAFGRDGSIFVNGYKYFSLPPIKSVNFSIGSVEQHGFWTIAYTNYTDVMVDINGTGITRLFYCDSPINRIKCQQLKHELPDGFYAASMLVKKDLPKTFVTMPQFYNWMTVTLHVVLNDTEKKADIILAKAPELASLADIHFEIVQANGSVANVTSLCVQTRQIALFYKYTSLQSLYTYSNLVELQNYDCPFSPQQFNNYLQFETLCLDTNPAVAGCKWALVHDVKWRTQFATITVSYKDGAMITTMP

SEQ ID NO: 6 - protein, 349 aa, synthetic construct
FGKTMHSYFCFANFSNSMVRQFLGILPPTVREFAFGRDGSIFVNGYKYFSLPPIKSVNFSIGSVEQHGFWTIAYTNYTDVMVDINGTGITRLFYCDSPINRIKCQQLKHELPDGFYAASMLVKKDLPKTFVTMPQFYNWMTVTLHVVLNDTEKKADIILAKAPELASLADIHFEIVQANGSVANVTSLCVQTRQIALFYKYTSLQSLYTYSNLVELQNYDCPFSPQQFNNYLQFETLCLDTNPAVAGCKWALVHDVKWRTQFATITVSYKDGAMITTMPGAGSSLEVLFQGPGSGSSHHHHHHHHGGSGSSMDEKTTGWRGGHVVEGLAGELEQLRARLEHHPQGQREP

SEQ ID NO: 7 - protein, 268 aa, synthetic construct
NGTALKYLGTLPPSVKEIAISKWGHFYINGYNFFSTFPIGCISFNLTTGVSGAFWTIAYTSYTEALVQVENTAIKNVTYCNSHINNIKCSQLTANLNNGFYPVASSEVGFVNKSVVLLPSFFTYTAVNITIDLGMKLSGYGQPIASTLSNITLPMQDNNTDVYCIRSNQFSVYVHSTCKSSLWDNIFNQDCTDVLEATAVIKTGTCPFSFDKLNNYLTFNKFCLSLSPVGANCKFDVAARTRTNEQVVRSLYVIYEEGDNIVGVPSDN

SEQ ID NO: 8 - protein, 338 aa, synthetic construct
NGTALKYLGTLPPSVKEIAISKWGHFYINGYNFFSTFPIGCISFNLTTGVSGAFWTIAYTSYTEALVQVENTAIKNVTYCNSHINNIKCSQLTANLNNGFYPVASSEVGFVNKSVVLLPSFFTYTAVNITIDLGMKLSGYGQPIASTLSNITLPMQDNNTDVYCIRSNQFSVYVHSTCKSSLWDNIFNQDCTDVLEATAVIKTGTCPFSFDKLNNYLTFNKFCLSLSPVGANCKFDVAARTRTNEQVVRSLYVIYEEGDNIVGVPSDNGAGSSLEVLFQGPGSGSSHHHHHHHHGGSGSSMDEKTTGWRGGHVVEGLAGELEQLRARLEHHPQGQREP

SEQ ID NO: 9 - protein, 277 aa, synthetic construct
RTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFHHHHHHHH

SEQ ID NO: 10 - protein, 211 aa, synthetic construct; identical to SEQ ID NO: 1

SEQ ID NO: 11 - protein, 281 aa, synthetic construct
FGSTDDPYLCFVNFSTAALDNQFLGILPPTIREMAFGRDGSIFINGYKYFNMPPIKSINFAISAVEYYGFWTIAYTKYTDVMVDVNGTSITRLFYWDSPLNRIKGQQMKHDLPDGFYSANSLVKKDLPKTFVTTPQFYNWMNVTLHVILNDTEAGYKVVGFERYELEKIADIHFEIPQDKNGGVKNVTNFCVQTRQLAFFFKYTSLQGLYSISNTVWLKNYDCPFSPQQFNNYLQFETLCFDTNPSVAGCKWAIMHSPIWNTQFATITVTYKEGAQITTMP

SEQ ID NO: 12 - protein, 268 aa, synthetic construct
NGTALKYLGTLPPSVKEIAISKWGHFYINGYNFFSTFPIDCISFNLTTGDSGAFWTIAYTSYTDALVQVENTAIKKVTYCNSHINNIKCSQLTANLQNGFYPVASSEVGLVNKSVVLLPTFYSHTSFNITIDLGMKRSGYGQPIASTLSNITLPMQDNNTDVYCVRSNRFSVYFHSTCKSSSWDDVFNSDCTDVLYATAVIKTGTCPFSFDKLNNYLTFNKFCLSLNPVGANCKFDVAARTRTNEQVVRSLYVIYEEGDNIVGVPSDN

SEQ ID NO: 13 - protein, 11 aa, synthetic construct
DLSPRWYFYYL

SEQ ID NO: 14 - protein, 10 aa, synthetic construct
MEVTPSGTWL

SEQ ID NO: 15 - protein, 9 aa, synthetic construct
QKKQQTVTL

SEQ ID NO: 16 - protein, 9 aa, synthetic construct
ELAERWFFY

SEQ ID NO: 17 - protein, 9 aa, synthetic construct
EEAGDQVKV

SEQ ID NO: 18 - protein, 9 aa, synthetic construct
KPEELSVTL

SEQ ID NO: 19 - protein, 9 aa, synthetic construct
RQRLTKYTM

SEQ ID NO: 20 - protein, 9 aa, synthetic construct
VENPDILRV

SEQ ID NO: 21 - protein, 9 aa, synthetic construct
NQDLNGNWY

SEQ ID NO: 22 - protein, 9 aa, synthetic construct
AESHVDTDL

SEQ ID NO: 23 - protein, 12 aa, synthetic construct
ERLKLFDRYFKY

SEQ ID NO: 24 - protein, 28 aa, synthetic construct
FPPTSFGPLVRKIFVDGVPFVVSTGYHF

SEQ ID NO: 25 - protein, 9 aa, synthetic construct
YAADPAMHA

SEQ ID NO: 26 - protein, 11 aa, synthetic construct
FQTVKPGNFNK

SEQ ID NO: 27 - protein, 9 aa, synthetic construct
VELKHFFFA

SEQ ID NO: 28 - protein, 15 aa, synthetic construct
ISDYDYYRYNLPTMC

SEQ ID NO: 29 - protein, 10 aa, synthetic construct
KSAGFPFNKW

SEQ ID NO: 30 - protein, 17 aa, synthetic construct
RLYYDSMSYEDQDALFA

SEQ ID NO: 31 - protein, 9 aa, synthetic construct
IPTITQMNL

SEQ ID NO: 32 - protein, 9 aa, synthetic construct
RQFHQKLLK

SEQ ID NO: 33 - protein, 9 aa, synthetic construct
ATRGATVVI

SEQ ID NO: 34 - protein, 9 aa, synthetic construct
LRIMASLVL

SEQ ID NO: 35 - protein, 9 aa, synthetic construct
RLANECAQV

SEQ ID NO: 36 - protein, 9 aa, synthetic construct
SSGDATTAY

SEQ ID NO: 37 - protein, 9 aa, synthetic construct
SVFNICQAV

SEQ ID NO: 38 - protein, 9 aa, synthetic construct
VRNLQHRLY

SEQ ID NO: 39 - protein, 9 aa, synthetic construct
FVNEFYAYL

SEQ ID NO: 40 - protein, 14 aa, synthetic construct
KNFKSVLYYQNNVF

SEQ ID NO: 41 - protein, 9 aa, synthetic construct
HEFCSQHTM

SEQ ID NO: 42 - protein, 9 aa, synthetic construct
LPYPDPSRI

SEQ ID NO: 43 - protein, 18 aa, synthetic construct
LMIERFVSLAIDAYPLTK

SEQ ID NO: 44 - protein, 9 aa, synthetic construct
RKDLTKYTM

SEQ ID NO: 45 - protein, 9 aa, synthetic construct
VENEAIHEV

SEQ ID NO: 46 - protein, 9 aa, synthetic construct
NQDLNGNFY

SEQ ID NO: 47 - protein, 9 aa, synthetic construct
SENFVKSDI

SEQ ID NO: 48 - protein, 12 aa, synthetic construct
HKEKLFHKYFKH

SEQ ID NO: 49 - protein, 26 aa, synthetic construct
IPSTAFGPLVRKVHIDGVPVVVTAGY

SEQ ID NO: 50 - protein, 9 aa, synthetic construct
FVTDPTLLV

SEQ ID NO: 51 - protein, 11 aa, synthetic construct
YQTVKPGHFNK

SEQ ID NO: 52 - protein, 9 aa, synthetic construct
SELTLKHFF

SEQ ID NO: 53 - protein, 15 aa, synthetic construct
MTDFNYYRYNRVTVL

SEQ ID NO: 54 - protein, 9 aa, synthetic construct
KSAGYPLNK

SEQ ID NO: 55 - protein, 18 aa, synthetic construct
RLYYETLSYEEQDALFAL

SEQ ID NO: 56 - protein, 9 aa, synthetic construct
LPTMTQMNL

SEQ ID NO: 57 - protein, 9 aa, synthetic construct
RQYHQKHLK

SEQ ID NO: 58 - protein, 9 aa, synthetic construct
ATRNATVVI

SEQ ID NO: 59 - protein, 9 aa, synthetic construct
IRMASAMIL

SEQ ID NO: 60 - protein, 9 aa, synthetic construct
RLSNELAQV

SEQ ID NO: 61 - protein, 9 aa, synthetic construct
TSGDGTTAY

SEQ ID NO: 62 - protein, 18 aa, synthetic construct
SVFNICQAVVKSIQRKIY

SEQ ID NO: 63 - protein, 9 aa, synthetic construct
FVVEYFSYL

SEQ ID NO: 64 - protein, 14 aa, synthetic construct
NAFKATLYYQNNVF

SEQ ID NO: 65 - protein, 9 aa, synthetic construct
HEFCSQHTL

SEQ ID NO: 66 - protein, 9 aa, synthetic construct
LPYPDPSRI

SEQ ID NO: 67 - protein, 18 aa, synthetic construct
IMLERYVSLAIDAYPLTK

SEQ ID NO: 68 - protein, 9 aa, synthetic construct
QVTCGTTTL

SEQ ID NO: 69 - protein, 9 aa, synthetic construct
DVVYCPRHV

SEQ ID NO: 70 - protein, 24 aa, synthetic construct
NPKTPKYKFVRIQPGQTFSVLACY

SEQ ID NO: 71 - protein, 9 aa, synthetic construct
AMRPNFTIK

SEQ ID NO: 72 - protein, 9 aa, synthetic construct
FLNGSCGSV

SEQ ID NO: 73 - protein, 18 aa, synthetic construct
MELPTGVHAGTDLEGNFY

SEQ ID NO: 74 - protein, 27 aa, synthetic construct
TVNVLAWLYAAVINGDRWFLNRFTTTL

SEQ ID NO: 75 - protein, 9 aa, synthetic construct
DEFTPFDVV

SEQ ID NO: 76 - protein, 9 aa, synthetic construct
RVAYGNNVL

SEQ ID NO: 77 - protein, 9 aa, synthetic construct
EVICPRHVI

SEQ ID NO: 78 - protein, 24 aa, synthetic construct
NPNTPEHKFKSVRPGESFNILACY

SEQ ID NO: 79 - protein, 9 aa, synthetic construct
NMRSQGTIK

SEQ ID NO: 80 - protein, 9 aa, synthetic construct
FIAGTCGSV

SEQ ID NO: 81 - protein, 18 aa, synthetic construct
LELGNGSHVGSNLEGEMY

SEQ ID NO: 82 - protein, 27 aa, synthetic construct
SDNVVAFLYAALINGERWFVTNTSMTL

SEQ ID NO: 83 - protein, 9 aa, synthetic construct
DEFTPIEVI

SEQ ID NO: 84 - protein, 5 aa, synthetic construct
KVSVR

SEQ ID NO: 85 - protein, 15 aa, synthetic construct
TPKYKFVRIQPGQTF

SEQ ID NO: 86 - protein, 18 aa, synthetic construct
LAWLYAAVINGDRWFLNR

SEQ ID NO: 87 - protein, 15 aa, synthetic construct
HKFKSVRPGESFNIL

SEQ ID NO: 88 - protein, 23 aa, synthetic construct
VAFLYAALINGERWFVTNTSMTL

SEQ ID NO: 89 - protein, 75 aa, synthetic construct
KVSVRQVTCGTTTLKVSVRDVVYCPRHVKVSVRNPKTPKYKFVRIQPGQTFSVLACYKVSVRMRPNFTIKKVSVR

SEQ ID NO: 90 - protein, 88 aa, synthetic construct
KVSVRFLNGSCGSVKVSVRMELPTGVHAGTDLEGNFYKVSVRTVNVLAWLYAAVINGDRWFLNRFTTTLKVSVRDEFTPFDVVKVSVR

SEQ ID NO: 91 - protein, 76 aa, synthetic construct
KVSVRRVAYGNNVLKVSVREVICPRHVIKVSVRNPNTPEHKFKSVRPGESFNILACYKVSVRNMRSQGTIKKVSVR

SEQ ID NO: 92 - protein, 88 aa, synthetic construct
KVSVRFIAGTCGSVKVSVRLELGNGSHVGSNLEGEMYKVSVRSDNVVAFLYAALINGERWFVTNTSMTLKVSVRDEFTPIEVIKVSVR

SEQ ID NO: 93 - protein, 411 aa, synthetic construct
VQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLYENQKLIANQFNSAIGKIQDSLSSTASALGKLQDVVNQNAQALNTLVKQLPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 94 - protein, 496 aa, synthetic construct
VRQFLGILPPTVREFAFGRDGSIFVNGYKYFSLPPIKSVNFSIGSVEQHGFWTIAYTNYTDVMVDINGTGITRLFYCDSPINRIKCQQLKHELPDGFYAASMLVKKDLPKTFVTMPQFYNWMTVTLHVVLNDTEKKADIILAKAPELASLADIHFEIVQANGSVANVTSLCVQTRQIALFYKYTSLQSLYTYSNLVELQNYDCPFSPQQFNNYLQFETLCLDTNPAVAGCKWALVHDVKWRTQFATITVSYKDGAMITTMPKAQLGFQDISNLVKDECTDYNIYGLQENQKILANAFNNAIGNITLALGKVSNAITTISDGFNTMASALTKIQSVVNQQGEALSLLTSQLDYIDINKTIADMLEQYYSNYTTPELDLQLEIFNQTKLNLTAEIDQLEQRADNLTNIAHELQEYIDNLNKTLVDLEWLNRIETYVKWPWYVWLLIGLVVVFCIPLLLFCCLSTGCCGCFGCLGSCCHSLCSRRQFENYEPIEKVHIH

SEQ ID NO: 95 - protein, 498 aa, synthetic construct
NGTALKYLGTLPPSVKEIAISKWGHFYINGYNFFSTFPIGCISFNLTTGVSGAFWTIAYTSYTEALVQVENTAIKNVTYCNSHINNIKCSQLTANLNNGFYPVASSEVGFVNKSVVLLPSFFTYTAVNITIDLGMKLSGYGQPIASTLSNITLPMQDNNTDVYCIRSNQFSVYVHSTCKSSLWDNIFNQDCTDVLEATAVIKTGTCPFSFDKLNNYLTFNKFCLSLSPVGANCKFDVAARTRTNEQVVRSLYVIYEEGDNIVGVPSDNSGLHDLSVLHLDSCTDYNIYGLNKNQQILANAFNQAIGNITQAFGKVNDAIHQTSQGLATVAKALAKVQDVVNTQGQALSHLTVQLDYIDINQTVQDILENYEPNWTVPEFTLDIFNATYLNLTGEIDDLEFRSEKLHNTTVELAILIDNINNTLVNLEWLNRIETYVKWPWYVWLLIGLVVVFCIPLLLFCCFSTGCCGCIGCLGSCCHSICSRRQFENYEPIEKVHVH

SEQ ID NO: 96 - protein, 422 aa, synthetic construct
VQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLYENQKLIANQFNSAIGKIQDSLSSTASALGKLQDVVNQNAQALNTLVKQLPLQPELDSFKEELDKYFKNHTSPNFDLTPFNLTYLNLSSELKQLEAKTASLFQTTVELQGLIDQINSTYVDLKLLGKYEQYIKWPWYIWLGFIAGLIAIVMVTIMLCCMTSCCSCLKGCCSCGSCCKFDEDDSEPVLKGVKLHYT

SEQ ID NO: 97 - protein, 424 aa, synthetic construct
VQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLYENQKLIANQFNSAIGKIQDSLSSTASALGKLQDVVNQNAQALNTLVKQLPLQPELDSFKEELDKYFKNHTSPELDLQLEIFNQTKLNLTAEIDQLEQRADNLTNIAHELQEYIDNLNKTLVDLEWLGKYEQYIKWPWYIWLGFIAGLIAIVMVTIMLCCMTSCCSCLKGCCSCGSCCKFDEDDSEPVLKGVKLHYT

SEQ ID NO: 98 - protein, 427 aa, synthetic construct
VQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLYENQKLIANQFNSAIGKIQDSLSSTASALGKLQDVVNQNAQALNTLVKQLHRHPLQPELDSFKEELDKYFKNHTSPELDLQLEIFNQTKLNLTAEIDQLEQRADNLTNIAHELQEYIDNLNKTLVDLEWLGKYEQYIKWPWYIWLGFIAGLIAIVMVTIMLCCMTSCCSCLKGCCSCGSCCKFDEDDSEPVLKGVKLHYT